(19) United States
(12) Patent Application Publication
(10) Pub. No.: US 2025/0073175 A1
(43) Pub. Date: Mar. 6, 2025

(54) Intra-Dosage Form Coatings and Applications Thereof

(71) Applicant: Balchem Corporation, New Hampton, NY (US)

(72) Inventors: Clayton ERICSON, Hampton, NY (US); Ren A. GONZALEZ, New Hampton, NY (US)

(21) Appl. No.: 18/845,997

(22) PCT Filed: Mar. 10, 2023
(86) PCT No.: PCT/US2023/064173

§ 371 Date: Sep. 11, 2024

Related U.S. Application Data
(60) Provisional application No. 63/318,946, filed on Mar. 11, 2022.

(51) Int. Cl. A61K 9/50; A61K 9/20; A61K 31/14; A61K 31/375

(52) U.S. Cl. CPC A61K 9/5015; A61K 9/2081; A61K 31/14; A61K 31/375

(57) ABSTRACT

The present disclosure relates to coating formulations for use in multi-ingredient dosage forms comprising vitamin C and/or choline. The coating formulations surround the vitamin C and/or the choline in a multi-ingredient dosage form, which prevents spotting of the dosage forms.

### CROSS-REFERENCE TO RELATED APPLICATIONS

This application claims priority to U.S. Provisional Application No. 63/318,946 entitled “INTRA-DOSAGE FORM COATINGS AND APPLICATIONS THEREOF”, filed on Mar. 11, 2022, the entire contents of which are incorporated by reference herein.

### FIELD OF THE DISCLOSURE

The present disclosure relates to coating formulations and methods of use and making thereof. Accordingly, the present disclosure relates to the fields of chemistry, pharmacy, and dietary supplementation.

### BACKGROUND

Choline is an essential nutrient that is present in many foods. Although choline is available in dietary supplements, it is rarely included in dosage forms such as multivitamins that include vitamin C. This is generally due to the hygroscopic nature of choline. When vitamin C degrades, it creates black spots on the surface of the dosage form that makes it unappealing to consumers. Furthermore, this also reduces the potency of vitamin C in the dosage form, forcing manufacturers to either overformulate the dosage form or risk failing the label claim of vitamin C.

When choline is present in the dosage form, the choline absorbs moisture from the air or from other components in the dosage form, accelerating the degradation of vitamin C. Additionally, the presence of metals and metal oxides in the dosage form accelerates the degradation of vitamin C. Moreover, other agents in the dosage form can also accelerate spotting, such as magnesium oxide, ferrous fumarate, and dicalcium phosphate. This accelerates the spotting caused by vitamin C degradation.

What is needed is a dosage form comprising vitamin C and, optionally, choline that reduces the rate of degradation of vitamin C to prevent spotting.

### SUMMARY OF THE DISCLOSURE

Provided herein are coating formulations comprising shellac that, when coated on at least one active ingredient in a multi-ingredient dosage form, prevent spotting in the dosage form. In some embodiments, the at least one active ingredient has a concentration of between about 50.0 mass % to about 95.0 mass % relative to the total mass of the at least one active ingredient and the coating formulation.

In some embodiments, the at least one active ingredient comprises vitamin C. In some aspects, the vitamin C is selected from the group consisting of ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, silver ascorbate, and any combination thereof. In some additional aspects, the vitamin C is present in an amount of between about 50 mass % to about 95 mass % relative to the combined mass of the vitamin C and the coating formulation.

In some embodiments, the at least one active ingredient comprises choline. In some embodiments, the choline is selected from the group consisting of choline chloride, choline bitartrate, phosphatidyl choline, choline dihydrogen citrate, alpha glycerylphosphorylcholine, cytidine diphosphate-choline, choline folate, an ester of choline with DHA, esters of choline with 3-methyl-2-hexenoic acid (MHA), esters of choline with medium chain triglycerides, other esters of choline with fatty acids, other salts of choline, or combinations thereof. In some additional aspects, the choline is present in an amount of between about 50 mass % to about 95 mass % relative to the combined mass of the choline and the coating formulation.

In some embodiments, the shellac has a concentration of between about 1 mass % to about 50 mass % relative to the combined mass of the at least one active ingredient and the coating formulation. In exemplary embodiments, the coating formulation coating formulation further comprises at least one of hydroxypropylcellulose, silicon dioxide, starch, microcrystalline cellulose, ethylcellulose, talc, and combinations thereof.

In some aspects, the coating formulation further comprises a silicate. In some further aspects, the silicate includes silicon dioxide, bentonite, magnesium aluminum silicate, magnesium silicate, magnesium trisilicate, silicified microcrystalline cellulose, or combinations thereof. In some examples, the silicate includes silicon dioxide, and the silicon dioxide is present in the coating formulation in an amount of about 0 mass % to about 10 mass % relative to the combined mass of the coating formulation and the at least one active ingredient. In still further examples, the silicon dioxide is at least one of colloidal silicon dioxide or fumed silica.

In some aspects, the coating formulation includes cellulose or a derivative thereof. In some additional aspects, the cellulose or a derivative thereof includes microcrystalline cellulose, powdered cellulose, hydroxypropylcellulose, hydroxypropylmethylcellulose, hydroxypropylcellulose, methylcellulose, ethylcellulose, hydroxyethylcellulose, hydroxyethylmethylcellulose, hypromellose, carboxymethylcellulose, or combinations thereof.

In some aspects, the coating formulation further comprises hydroxypropylcellulose. In some additional aspects, the hydroxypropylcellulose is present in the coating formulation in an amount of about 0 mass % to about 10 mass % relative to the combined mass of the coating formulation and the at least one active ingredient.

In some aspects, the coating formulation further comprises microcrystalline cellulose. In some additional aspects, the microcrystalline cellulose is present in the coating formulation in an amount of about 0 mass % to about 25 mass % relative to the combined mass of the coating formulation and the first active ingredient and second active ingredient.

In some aspects, the coating formulation further comprises ethylcellulose. In some additional aspects, the ethylcellulose is present in the coating formulation in an amount of about 0 mass % to about 10 mass % relative to the combined mass of the coating formulation and the first active ingredient or the second active ingredient.

In some aspects, the coating formulation further comprises starch. In some further aspects, the starch includes maize starch, potato starch, rice starch, wheat starch, tapioca starch, or combinations thereof. In some additional aspects, the starch is present in the coating formulation in an amount of about 0 mass % to about 25 mass % relative to the combined mass of the coating formulation and the first active ingredient or the second active ingredient.

Further provided herein is a multi-ingredient dosage form, the dosage form comprising at least one active ingredient and a coating formulation comprising shellac, wherein the coating formulation coats the at least one active ingredient. In some embodiments, the shellac is present in an amount between about 1 mass % to about 50 mass % relative to the total mass of the at least one active ingredient and the coating formulation. In some additional embodiments, the at least one active ingredient has a concentration of between about 50.0 mass % to about 95.0 mass % relative to the combined mass of the at least one active ingredient and the coating formulation.

In some embodiments, the at least one active ingredient comprises vitamin C. In some aspects, the vitamin C is selected from the group consisting of ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, silver ascorbate, and any combination thereof. In some additional aspects, the vitamin C is present in the dosage form in an amount of about 1 mg to about 2000 mg.

In some embodiments, the at least one active ingredient comprises choline. In some embodiments, the choline is selected from the group consisting of choline chloride, choline bitartrate, phosphatidyl choline, choline dihydrogen citrate, alpha glycerylphosphorylcholine, cytidine diphosphate-choline, choline folate, an ester of choline with DHA, esters of choline with 3-methyl-2-hexenoic acid (MHA), esters of choline with medium chain triglycerides, other esters of choline with fatty acids, other salts of choline, or combinations thereof. In some additional aspects, the choline is present in the dosage form in an amount of about 1 mg to about 2000 mg.

In some exemplary embodiments, the coating formulation further comprises a silicate, a starch, cellulose or a derivative thereof, or a combination thereof.

In an exemplary embodiment, the at least one active ingredient comprises vitamin C and choline, and both the vitamin C and the choline are coated by the coating formulation. In another exemplary embodiment, the at least one active ingredient comprises choline and the dosage form further comprises vitamin C, and the vitamin C is not coated by the coating formulation. In yet another exemplary embodiment, the at least one active ingredient comprises vitamin C and the dosage form further comprises choline, wherein the choline is not coated by the coating formulation.

In some embodiments, the multi-ingredient dosage form is a tablet, capsule, gummy, beverage, bar, suspension, granulation, powder, lozenge, troche, granule, pill, gel cap. In some embodiments, the dosage form further comprises at least one of vitamins, minerals, supplements, and excipients.

In some embodiments, the multi-ingredient dosage form, wherein the multi-ingredient dosage form shows little spotting after storage for about 18 days, 30 days, or 60 days at 25° C. and 20% relative humidity. In some embodiments, the multi-ingredient dosage form, wherein the multi-ingredient dosage form shows little spotting after storage for about 18 days, 30 days, or 60 days at 25° C. and 30% relative humidity. In some embodiments, the multi-ingredient dosage form, wherein the multi-ingredient dosage form shows little spotting after storage for about 18 days, 30 days, or 60 days at 45° C. and 20% relative humidity. In some embodiments, the multi-ingredient dosage form, wherein the multi-ingredient dosage form shows little spotting after storage for about 18 days, 30 days, or 60 days at 45° C. and 30% relative humidity.

Further provided herein is a method of preventing spotting in a dosage form. The method includes providing an active ingredient comprising at least one of vitamin C and choline; coating the active ingredient in a coating formulation comprising shellac; and including the active ingredient coated in the coating formulation in a dosage form.

### BRIEF DESCRIPTION OF THE FIGURES

FIG. 1 shows spotting for the tablet of Example 1 containing vitamin C but no choline under different storage conditions.

FIGS. 2A-2D show four different commercially available tablets stored under different conditions.

FIG. 3 shows spotting for the tablet of Example 1 containing choline and vitamin C under different storage conditions for 18 days. The temperature ranged from 25° C. to 85° C. and relative humidity ranged from 0% RH to 60% RH.

FIG. 4 shows spotting for the tablet of Example 1 containing choline and vitamin C under refrigerated storage conditions (4° C.) at 0% RH, 30% RH, and 60% RH, for 18 days.

FIGS. 5A and 5B show spotting under different storage conditions for the tablet of Example 1 containing various forms of choline and vitamin C.

FIGS. 6A and 6B show spotting under different storage conditions for the tablet of Example 1 containing various dosage forms, different forms of vitamin C, and choline.

FIG. 7 shows spotting under different storage conditions for the tablet of Example 1 including vitamin C, choline, and a coating formulation of the present disclosure comprising talc.

FIG. 8 shows progression of spotting under different storage conditions for the tablet of Example 1, including vitamin C, choline, and a coating formulation of the present disclosure comprising talc.

FIG. 9 shows the progression of spotting for tablets under different storage conditions, wherein the tablets contain vitamin C, choline, and a coating formulation of the present disclosure consisting of shellac.

FIG. 10 shows the progression of spotting for tablets of Example 1 under different storage conditions, wherein the tablets contain vitamin C, choline, and either a coating formulation of the present disclosure consisting of shellac or no coating formulation.

FIG. 11 shows the progression of spotting for tablets of Example 1 under different storage conditions, wherein the tablets contain a coating formulation of the present disclosure coated on choline, on vitamin C, or on both.

FIGS. 12A-12G show colorimeter diagrams for various tablets having various coating formulations of the present disclosure.

FIG. 13 shows the tablets and the ΔE values for various tablets containing various coating formulations of the present disclosure.

FIGS. 14A-14B show colorimeter diagrams for various tablets that do not contain a coating formulation of the present disclosure. FIG. 14C shows exemplary samples used in the colorimeter experiments.

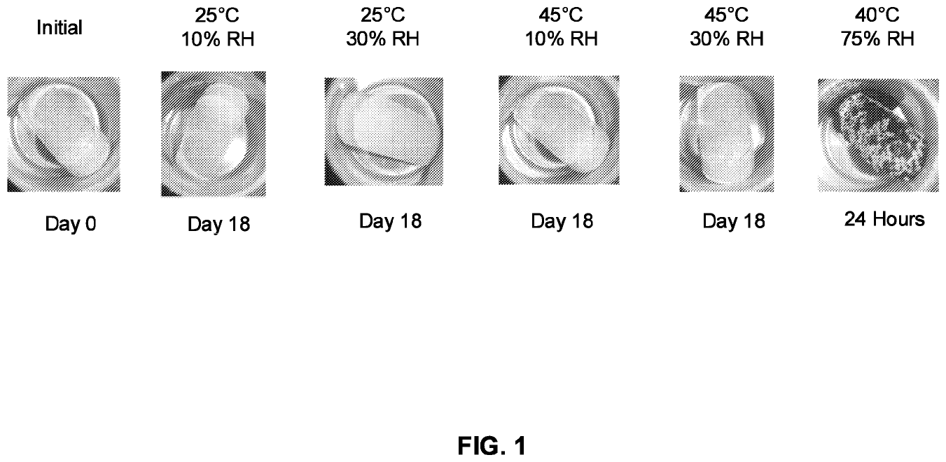

### DETAILED DESCRIPTION

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc. As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, the term “spotting” refers to the formation of black spots on a dosage form that contains vitamin C and, optionally, choline chloride.

As used herein, the term “choline” refers to both the N,N,N-trimethylethanolammonium cation as well as the various quarternary ammonium salts, phospholipid-bound forms of the cation, esters of the cation with fatty acids or medium chain triglycerides, and other molecules containing the cation. Thus, the term “choline” may be used interchangeably with “choline salt”, “phospholipid-bound forms of choline”, “esters of choline with fatty acids”, etc. However, when the term “choline salt” is used, it refers specifically to a salt form of the N,N,N-trimethylethanolammounium cation; when the term “esters of choline with medium chain triglycerides” is used, it refers specifically to an esterified form of the N,N,N-trimethylethanolammonium cation; and so on.

As used herein, the term “vitamin C” refers to both the ascorbate ion having the molecular formula C₆H₇O₆⁻ as well as the acid, salt, and oxidized forms of the ascorbate ion. Thus, the term “vitamin C” may be used interchangeably with “ascorbic acid”. When amounts or concentrations of vitamin C are referred to herein, they generally refer to the concentration or the amount of the ascorbate ion; for example, the phrase “the vitamin C may be present in the amount of 1 mg” generally means herein that the amount of the ascorbate ion is 1 mg.

As used herein, the term “shellac” refers to the resin secreted by the female lac bug. The term encompasses raw shellac, liquid shellac, and shellac that has been processed, modified, or purified through methods known to those having skill in the art, particularly through methods known for preparing shellac for pharmaceutical use. Moreover, the term includes derivatives or analogues of shellac.

Disclosed herein are coating formulations that are useful in preventing spotting in multi-ingredient dosage forms containing vitamin C and, optionally, choline chloride. The coating formulations comprise shellac. Without being bound by theory, the coating formulations may reduce the rate of degradation of vitamin C by protecting the vitamin C from moisture and reducing contact with other ingredients (e.g., metals and metal oxides) within the multi-ingredient dosage form. Additionally or alternatively, and without being bound by theory, the coating formulations may prevent choline chloride from absorbing excess moisture from the environment or from other components in the dosage form, which may accelerate the degradation of vitamin C. This also may have the added benefit of reducing the formation of metal oxides and reducing the interaction with metals in the multi-ingredient dosage form. Furthermore, the coating formulation is resistant to fracturing when compressed, allowing the coating formulation to be used in tablets and other dosage forms manufactured by compression.

### I. Composition

### A. Coating Formulation

Disclosed herein is a coating formulation useful in preventing spotting in dosage forms containing at least one active ingredient. The coating formulation includes shellac. In some examples, the coating formulation may consist of or consist essentially of shellac. The coating formulation surrounds an active ingredient. The coating formulation may completely surround the active ingredient or may substantially surround the active ingredient (i.e., 95% or more of the active ingredient). Preferably, the coating formulation does not coat the entire dosage form and only coats the active ingredients described below.

The particle size of the coated active ingredient (i.e., the coating formulation plus the active ingredient) may be from about 1 micron to about 2000 microns. In some aspects, the particle size of the coated active ingredient may be from about 1 micron to about 50 microns, about 50 microns to about 100 microns, about 100 microns to about 250 microns, about 250 microns to about 500 microns, about 500 microns to about 750 microns, about 750 microns to about 1000 microns, about 1000 microns to about 1250 microns, about 1250 microns to about 1500 microns, about 1500 microns to about 1750 microns, or about 1750 microns to about 2000 microns. In some additional aspects, the particle size of the coated active ingredient may be from about 1 micron to about 100 microns, about 1 micron to about 250 microns, about 1 micron to about 500 microns, about 1 micron to about 750 microns, about 1 micron to about 1000 microns, about 1 micron to about 1250 microns, about 1 micron to about 1500 microns, about 1 micron to about 1750 microns, about 50 microns to about 2000 microns, about 100 microns to about 2000 microns, about 250 microns to about 2000 microns, about 500 microns to about 2000 microns, about 750 microns to about 2000 microns, about 1000 microns to about 2000 microns, about 1250 microns to about 2000 microns, or about 1500 microns to about 2000 microns. In still further embodiments, the particle size of the coated active ingredient may be about 1 micron, 50 microns, 100 microns, 200 microns, 300 microns, 400 microns, 500 microns, 600 microns, 700 microns, 800 microns, 900 microns, 1000 microns, 1100 microns, 1200 microns, 1300 microns, 1400 microns, 1500 microns, 1600 microns, 1700 microns, 1800 microns, 1900 microns, or about 2000 microns.

The coating formulation may be present in an amount from about 5 mass % to about 50 mass % relative to the combined mass of the at least one active ingredient and the coating formulation. In some aspects, the coating formulation may be present in an amount from about 5 mass % to about 10 mass %, about 5 mass % to about 15 mass %, about 5 mass % to about 20 mass %, about 5 mass % to about 25 mass %, about 5 mass % to about 30 mass %, about 5 mass % to about 35 mass %, about 5 mass % to about 40 mass %, about 5 mass % to about 45 mass %, about 45 mass % to about 50 mass %, about 40 mass % to about 50 mass %, about 35 mass % to about 50 mass %, about 30 mass % to about 50 mass %, about 25 mass % to about 50 mass %, about 2 mass % to about 50 mass %, about 15 mass % to about 50 mass %, or about 10 mass % to about 50 mass % relative to the combined mass of the at least one active ingredient and the coating formulation. In some additional aspects, the coating formulation may be present in an amount of about 5 mass %, 7.5 mass %, 10 mass %, 12.5 mass %, 15 mass %, 17.5 mass %, 20 mass %, 22.5 mass %, 25 mass %, 27.5 mass %, 30 mass %, 32.5 mass %, 35 mass %, 37.5 mass %, 40 mass %, 42.5 mass %, 45 mass %, 47.5 mass %, or about 50 mass % relative to the combined mass of the at least one active ingredient and the coating formulation.

The shellac may be present in the coating formulation in an amount from about 1 mass % to about 50 mass % relative to the combined mass of the at least one active ingredient and the coating formulation. In some aspects, the shellac may be present in an amount from about 1 mass % to about 5 mass %, about 1 mass % to about 10 mass %, about 1 mass % to about 15 mass %, about 1 mass % to about 20 mass %, about 1 mass % to about 25 mass %, about 1 mass % to about 30 mass %, about 1 mass % to about 35 mass %, about 1 mass % to about 40 mass %, about 1 mass % to about 45 mass %, about 45 mass % to about 50 mass %, about 40 mass % to about 50 mass %, about 35 mass % to about 50 mass %, about 30 mass % to about 50 mass %, about 25 mass % to about 50 mass %, about 2 mass % to about 50 mass %, about 15 mass % to about 50 mass %, about 10 mass % to about 50 mass %, about 5 mass % to about 50 mass %, about 5 mass % to about 40 mass %, about 5 mass % to about 30 mass %, about 5 mass % to about 20 mass %, about 10 mass % to about 40 mass %, about 10 mass % to about 30 mass %, or about 10 mass % to about 20 mass % relative to the combined mass of the at least one active ingredient and the coating formulation. In some additional aspects, the shellac may be present in an amount from about 1 mass %, 2.5 mass %, 5 mass %, 7.5 mass %, 10 mass %, 12.5 mass %, 15 mass %, 17.5 mass %, 20 mass %, 22.5 mass %, 25 mass %, 27.5 mass %, 30 mass %, 32.5 mass %, 35 mass %, 37.5 mass %, 40 mass %, 42.5 mass %, 45 mass %, 47.5 mass %, or about 50 mass % relative to the combined mass of the at least one active ingredient and the coating formulation. In some examples, the shellac may be present in an amount from about 5 mass % to about 20 mass % relative to the combined mass of the at least one active ingredient and the coating formulation.

The coating formulation may include a silicate, such as silicon dioxide, bentonite, magnesium aluminum silicate, magnesium silicate, magnesium trisilicate, silicified microcrystalline cellulose, and other silicates known in the art or combinations thereof. The silicate acts as an adsorbent, an anti-caking agent, a glidant, a disintegrant, etc. The silicon dioxide may include colloidal silicon dioxide, fumed (pyrogenated) silica, or other forms of silicon dioxide. The silicate may be present in the coating formulation in an amount from about 0 mass % to about 10 mass % relative to the combined mass of the coating formulation and the at least one active ingredient. In some aspects, the silicate may be present in the coating formulation in an amount from about 0 mass % to about 2 mass %, 0 mass % to about 3 mass %, about 0 mass % to about 4 mass %, about 0 mass % to about 5 mass %, about 0 mass % to about 6 mass %, about 0 mass % to about 7 mass %, 0 mass % to about 8 mass %, about 0 mass % to about 9 mass %, about 1 mass % to about 10 mass %, about 2 mass % to about 10 mass %, about 3 mass % to about 10 mass %, about 4 mass % to about 10 mass %, about 5 mass % to about 10 mass %, about 6 mass % to about 10 mass %, about 7 mass % to about 10 mass %, about 8 mass % to about 10 mass %, about 0.5 mass % to about 5 mass %, or about 1 mass % to about 3 mass % relative to the combined mass of the coating formulation and the at least one active ingredient. In some additional aspects, the silicate may be present in the coating formulation in an amount from about 0 mass % to about 1 mass %, about 1 mass % to about 2 mass %, about 2 mass % to about 3 mass %, about 3 mass % to about 4 mass %, about 4 mass % to about 5 mass %, about 5 mass % to about 6 mass %, about 6 mass % to about 7 mass %, about 7 mass % to about 8 mass %, about 8 mass % to about 9 mass %, or about 9 mass % to about 10 mass % relative to the combined mass of the coating formulation and the at least one active ingredient. In yet additional aspects, the silicate may be present in the coating formulation in an amount of about 0.5 mass %, 1 mass %, 1.5 mass %, 2 mass %, 2.5 mass %, 3 mass %, about 4 mass %, about 5 mass %, about 6 mass %, about 7 mass %, about 8 mass %, about 9 mass %, or about 10 mass % relative to the combined mass of the coating formulation and the at least one active ingredient. In an exemplary embodiment, the silicate is present in the coating formulation in an amount from about 0.5 mass % to about 3 mass % relative to the combined mass of the coating formulation and the at least one active ingredient. In another exemplary embodiment, the silicate is colloidal silicon dioxide.

The coating formulation may include starch, such as maize starch, potato starch, rice starch, wheat starch, tapioca starch, and other starches or combinations thereof. The starch acts as a lubricant and a low-solubility coating. The starch may be present in the coating formulation in an amount from about 0 mass % to about 25 mass % relative to the combined mass of the coating formulation and the at least one active ingredient. In some aspects, the starch may be present in the coating formulation in an amount from about 0 mass % to about 5 mass %, about 5 mass % to about 10 mass %, about 10 mass % to about 15 mass %, about 15 mass % to about 20 mass %, or about 20 mass % to about 25 mass % relative to the combined mass of the coating formulation and the at least one active ingredient. In some additional aspects, the starch may be present in the coating formulation in an amount from about 0 mass % to about 10 mass %, about 0 mass % to about 15 mass %, about 0 mass % to about 20 mass %, about 5 mass % to about 25 mass %, about 10 mass % to about 25 mass %, about 15 mass % to about 25 mass %, or about 5 mass % to about 20 mass %. In still further aspects, the starch may be present in the coating formulation in an amount of about 0 mass %, 1 mass %, 2 mass %, 3 mass %, 4 mass %, 5 mass %, 6 mass %, 7 mass %, 8 mass %, 9 mass %, 10 mass %, 11 mass %, 12 mass %, 13 mass %, 14 mass %, 15 mass %, 16 mass %, 17 mass %, 18 mass %, 19 mass %, 20 mass %, 21 mass %, 22 mass %, 23 mass %, 24 mass %, or about 25 mass % relative to the combined mass of the coating formulation and the at least one active ingredient. In an exemplary embodiment, the starch is present in the coating formulation in an amount from about 9 mass % to about 16 mass % relative to the combined mass of the coating formulation and the at least one active ingredient.

The coating formulation may include cellulose or derivatives thereof, such as microcrystalline cellulose, powdered cellulose, hydroxypropylcellulose, hydroxypropylmethylcellulose, hydroxypropylcellulose, methylcellulose, ethylcellulose, hydroxyethylcellulose, hydroxyethylmethylcellulose, hypromellose (e.g., hypromellose acetate succinate), carboxymethylcellulose (e.g., sodium carboxymethylcellulose), and combinations thereof. In some aspects, the cellulose may be blended with other ingredients, such as carrageenan, phosphate salts (e.g., calcium phosphate), gums, (e.g., guar gum), mannitol, silicates, etc.

In some embodiments, the coating formulation may include microcrystalline cellulose. Microcrystalline cellulose acts as an adsorbent, compression aide, diluent, binder, and a disintegrant in the dosage forms described herein. The microcrystalline cellulose may be present in the coating formulation in an amount from about 0 mass % to about 25 mass % relative to the combined mass of the coating formulation and the at least one active ingredient. In some aspects, the microcrystalline cellulose may be present in an amount from about 0 mass % to about 5 mass %, about 5 mass % to about 10 mass %, about 10 mass % to about 15 mass %, about 15 mass % to about 20 mass %, or about 20 mass % to about 25 mass % relative to the combined mass of the coating formulation and the at least one active ingredient. In some additional aspects, the microcrystalline cellulose may be present in the coating formulation in an amount from about 0 mass % to about 10 mass %, about 0 mass % to about 15 mass %, about 0 mass % to about 20 mass %, about 5 mass % to about 25 mass %, about 10 mass % to about 25 mass %, about 15 mass % to about 25 mass %, or about 5 mass % to about 20 mass %. In still further aspects, the microcrystalline cellulose may be present in an amount of about 0 mass %, 1 mass %, 2 mass %, 3 mass %, 4 mass %, 5 mass %, 6 mass %, 7 mass %, 8 mass %, 9 mass %, 10 mass %, 11 mass %, 12 mass %, 13 mass %, 14 mass %, 15 mass %, 16 mass %, 17 mass %, 18 mass %, 19 mass %, 20 mass %, 21 mass %, 22 mass %, 23 mass %, 24 mass %, or about 25 mass % relative to the combined mass of the coating formulation and the at least one active ingredient. In an exemplary embodiment, the microcrystalline cellulose is present in an amount of about 9 mass % to about 12 mass % relative to the combined mass of the coating formulation and the at least one active ingredient.

In some embodiments, the coating formulation may include hydroxypropylcellulose. Hydroxypropylcellulose adds flexibility to the coating. The hydroxypropylcellulose may be present in the coating formulation in an amount from about 0 mass % to about 10 mass % relative to the combined mass of the coating formulation and the at least one active ingredient. In some aspects, the hydroxypropylcellulose may be present in the coating formulation in an amount from about 0 mass % to about 2 mass %, 0 mass % to about 3 mass %, about 0 mass % to about 4 mass %, about 0 mass % to about 5 mass %, about 0 mass % to about 6 mass %, about 0 mass % to about 7 mass %, 0 mass % to about 8 mass %, about 0 mass % to about 9 mass %, about 1 mass % to about 10 mass %, about 2 mass % to about 10 mass %, about 3 mass % to about 10 mass %, about 4 mass % to about 10 mass %, about 5 mass % to about 10 mass %, about 6 mass % to about 10 mass %, about 7 mass % to about 10 mass %, about 8 mass % to about 10 mass %, about 0.5 mass % to about 5 mass %, or about 1 mass % to about 3 mass % relative to the combined mass of the coating formulation and the at least one active ingredient. In some additional aspects, the hydroxypropylcellulose may be present in the coating formulation in an amount from about 0 mass % to about 1 mass %, about 1 mass % to about 2 mass %, about 2 mass % to about 3 mass %, about 3 mass % to about 4 mass %, about 4 mass % to about 5 mass %, about 5 mass % to about 6 mass %, about 6 mass % to about 7 mass %, about 7 mass % to about 8 mass %, about 8 mass % to about 9 mass %, or about 9 mass % to about 10 mass % relative to the combined mass of the coating formulation and the at least one active ingredient. In still further aspects, the hydroxypropylcellulose may be present in the coating formulation in an amount of about 0.5 mass %, 1 mass %, 1.5 mass %, 2 mass %, 2.5 mass %, 3 mass %, about 4 mass %, about 5 mass %, about 6 mass %, about 7 mass %, about 8 mass %, about 9 mass %, or about 10 mass % relative to the combined mass of the coating formulation and the at least one active ingredient. In an exemplary embodiment, the hydroxypropylcellulose is present in the coating formulation in an amount from about 0.25 mass % to about 1.5 mass % relative to the combined mass of the coating formulation and the at least one active ingredient.

In some embodiments, the coating formulation may include hydroxypropylmethylcellulose. Hydroxypropylmethylcellulose adds flexibility to the coating. The hydroxypropylmethylcellulose may be present in the coating formulation in an amount from about 0 mass % to about 10 mass % relative to the combined mass of the coating formulation and the at least one active ingredient. In some aspects, the hydroxypropylmethylcellulose may be present in the coating formulation in an amount from about 0 mass % to about 2 mass %, 0 mass % to about 3 mass %, about 0 mass % to about 4 mass %, about 0 mass % to about 5 mass %, about 0 mass % to about 6 mass %, about 0 mass % to about 7 mass %, 0 mass % to about 8 mass %, about 0 mass % to about 9 mass %, about 1 mass % to about 10 mass %, about 2 mass % to about 10 mass %, about 3 mass % to about 10 mass %, about 4 mass % to about 10 mass %, about 5 mass % to about 10 mass %, about 6 mass % to about 10 mass %, about 7 mass % to about 10 mass %, about 8 mass % to about 10 mass %, about 0.5 mass % to about 5 mass %, or about 1 mass % to about 3 mass % relative to the combined mass of the coating formulation and the at least one active ingredient. In some additional aspects, the hydroxypropylmethylcellulose may be present in the coating formulation in an amount from about 0 mass % to about 1 mass %, about 1 mass % to about 2 mass %, about 2 mass % to about 3 mass %, about 3 mass % to about 4 mass %, about 4 mass % to about 5 mass %, about 5 mass % to about 6 mass %, about 6 mass % to about 7 mass %, about 7 mass % to about 8 mass %, about 8 mass % to about 9 mass %, or about 9 mass % to about 10 mass % relative to the combined mass of the coating formulation and the at least one active ingredient. In still further aspects, the hydroxypropylmethylcellulose may be present in the coating formulation in an amount of about 0.5 mass %, 1 mass %, 1.5 mass %, 2 mass %, 2.5 mass %, 3 mass %, about 4 mass %, about 5 mass %, about 6 mass %, about 7 mass %, about 8 mass %, about 9 mass %, or about 10 mass % relative to the combined mass of the coating formulation and the at least one active ingredient.

In some embodiments, the coating formulation may further comprise ethylcellulose. The ethylcellulose may be present in the coating formulation in an amount of from about 0 mass % to about 10 mass % relative to the combined mass of the coating formulation and the at least one active ingredient. In some aspects, the ethylcellulose may be present in the coating formulation in an amount from about 0 mass % to about 2 mass %, 0 mass % to about 3 mass %, about 0 mass % to about 4 mass %, about 0 mass % to about 5 mass %, about 0 mass % to about 6 mass %, about 0 mass % to about 7 mass %, 0 mass % to about 8 mass %, about 0 mass % to about 9 mass %, about 1 mass % to about 10 mass %, about 2 mass % to about 10 mass %, about 3 mass % to about 10 mass %, about 4 mass % to about 10 mass %, about 5 mass % to about 10 mass %, about 6 mass % to about 10 mass %, about 7 mass % to about 10 mass %, about 8 mass % to about 10 mass %, about 0.5 mass % to about 5 mass %, or about 1 mass % to about 3 mass % relative to the combined mass of the coating formulation and the at least one active ingredient. In some additional aspects, the ethylcellulose may be present in the coating formulation in an amount from about 0 mass % to about 1 mass %, about 1 mass % to about 2 mass %, about 2 mass % to about 3 mass %, about 3 mass % to about 4 mass %, about 4 mass % to about 5 mass %, about 5 mass % to about 6 mass %, about 6 mass % to about 7 mass %, about 7 mass % to about 8 mass %, about 8 mass % to about 9 mass %, or about 9 mass % to about 10 mass % relative to the combined mass of the coating formulation and the at least one active ingredient. In still additional aspects, the ethylcellulose may be present in the coating formulation in an amount of about 0.5 mass %, 1 mass %, 1.5 mass %, 2 mass %, 2.5 mass %, 3 mass %, about 4 mass %, about 5 mass %, about 6 mass %, about 7 mass %, about 8 mass %, about 9 mass %, or about 10 mass % relative to the combined mass of the coating formulation and the at least one active ingredient. In an exemplary embodiment, the ethylcellulose is present in the coating formulation in an amount from about 0.1 mass % to about 1 mass % relative to the combined mass of the coating formulation and the at least one active ingredient.

The coating formulation may further comprise one or more excipients. The one or more excipients may include sugar (e.g., lactose, dextrin, maltodextrin, glucose, sucrose, fructose, maltose, galactose, etc.), sugar alcohols (e.g., sorbitol, mannitol, xylitol, lactitol, isomalt, maltitol, erythritol, hydrogenated starch hydrolysates, etc.), other silicates (e.g., calcium silicate, magnesium silicate, and other forms of silicon dioxide), other derivatives of starch, other derivatives of cellulose (e.g., cellulose acetate, cellulose phthalate, etc.), alginates, stearates or stearic acid, dyes, colorants, flavoring agents, lubricants, binders, fillers, glidants, diluents, adsorbents, disintegrants, film-formers, compression aides, and other excipients known to those having skill in the art.

### B. Active Ingredient

The coating formulation coats at least one active ingredient contained within a multi-ingredient dosage form.

The at least one active ingredient may include vitamin C and choline. In some exemplary embodiments, the at least one active ingredient may include vitamin C only, choline only, or both vitamin C and choline. In some additional exemplary embodiments, the multi-ingredient dosage form may contain both vitamin C and choline, but only one of the active ingredients is coated with the coating formulation of the present disclosure. In still other exemplary embodiments, the multi-ingredient dosage form may contain both vitamin C and choline, which are both coated with the coating formulation of the present disclosure, but the composition of the coating formulation for the vitamin C differs from the composition of the coating formulation for the choline. In still other exemplary embodiments, the multi-ingredient dosage form may contain both vitamin C and choline, which are both coated with a coating formulation of an identical composition.

The at least one active ingredient may include fat-soluble vitamins or their derivatives, such as vitamin A, vitamin D, vitamin E, vitamin K, and other fat-soluble vitamins known in the art and derivatives thereof.

The at least one active ingredient may include water-soluble vitamins or their derivatives, such as folate, thiamine, riboflavin, niacin, biotin, pantothenic acid, pyridoxine, cobalamin, and other water-soluble vitamins known in the art and derivatives thereof.

The at least one active agent may include nutritionally relevant metals, such as sodium, potassium, calcium, magnesium, iron, copper, zinc, manganese, cobalt, chromium, and other nutritionally relevant metals known in the art and salts, complexes, or chelates thereof.

The at least one active ingredient may include a drug or pharmaceutical agent, such an analgesic agent, an anti-inflammatory agent, an anti-arrhythmic agent, an anti-asthma agent, an anti-bacterial agent, an anti-viral agent, an anti-coagulant, an anti-depressant, an anti-diabetic, an anti-epileptic, an anti-fungal agent, an anti-hypertensive agent, an anti-malarial, an anti-migraine agent, an anti-muscarinic agent, an anti-neoplastic agent, an immunosuppressant, an anti-protozoal agent, an anti-thyroid agent, an anxiolytic agent, a sedative, a hypnotic agent, a neuroleptic agent, a beta-Blocker, a cardiac inotropic agent, a corticosteroid, a diuretic agent, an anti-Parkinsonian agent, a gastrointestinal agent, an anti-histamine, a histamine-receptor antagonist, a lipid regulating agent, a muscle relaxant, nitrate and other anti-anginal agent, a nutritional agent, an opioid analgesic, or a stimulant.

i. Vitamin C

The at least one active ingredient may include vitamin C. The vitamin C may include ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, silver ascorbate, or any other forms of vitamin C known in the art or combinations thereof. In some examples, the vitamin C is ascorbic acid.

In some embodiments, the vitamin C may be present in an amount of between about 50 mass % to about 95 mass % relative to the combined mass of the vitamin C and the coating formulation. In some aspects, the vitamin C may be present in an amount from about 50 mass % to about 55 mass %, about 55 mass % to about 60 mass %, about 60 mass % to about 65 mass %, about 65 mass % to about 70 mass %, about 70 mass % to about 75 mass %, about 75 mass % to about 80 mass %, about 80 mass % to about 85 mass %, about 85 mass % to about 90 mass %, or about 90 mass % to about 95 mass % relative to the combined mass of the vitamin C and the coating formulation. In some additional aspects, the vitamin C may be present in an amount from about 50 mass % to about 60 mass %, about 50 mass % to about 65 mass %, about 50 mass % to about 70 mass %, about 50 mass % to about 75 mass %, about 50 mass % to about 80 mass %, about 50 mass % to about 85 mass %, about 50 mass % to about 90 mass %, about 55 mass % to about 95 mass %, about 60 mass % to about 95 mass %, about 65 mass % to about 95 mass %, about 70 mass % to about 95 mass %, about 75 mass % to about 95 mass %, about 80 mass % to about 95 mass %, or about 85 mass % to about 95 mass % relative to the combined weight of the vitamin C and the coating formulation. In still further aspects, the vitamin C may be present in an amount of about 50 mass %, 55 mass %, 60 mass %, 65 mass %, 70 mass %, 75 mass %, 80 mass %, 85 mass %, 90 mass %, or about 95 mass % relative to the combined mass of the vitamin C and the coating formulation. In some exemplary embodiments, the vitamin C may be present in an amount from about 70 mass % to about 80 mass % relative to the combined mass of the vitamin C and the coating formulation.

ii. Choline

In some embodiments, the at least one active ingredient may include choline. The choline may include choline chloride, choline bitartrate, phosphatidyl choline, choline dihydrogen citrate, alpha glycerylphosphorylcholine, cytidine diphosphate-choline, choline folate, an ester of choline with DHA, esters of choline with 3-methyl-2-hexenoic acid (MHA), esters of choline with medium chain triglycerides, other esters of choline with fatty acids, other salts of choline, or combinations thereof. In some examples, the choline is choline bitartrate.

In some embodiments, the choline may be present in an amount from about 50 mass % to about 95 mass % relative to the combined weight of the coating formulation and the choline. In some aspects, the choline may be present in an amount from about 50 mass % to about 55 mass %, about 55 mass % to about 60 mass %, about 60 mass % to about 65 mass %, about 65 mass % to about 70 mass %, about 70 mass % to about 75 mass %, about 75 mass % to about 80 mass %, about 80 mass % to about 85 mass %, about 85 mass % to about 90 mass %, or about 90 mass % to about 95 mass % relative to the combined mass of the coating formulation and the choline. In some additional aspects, the choline may be present in an amount from about 50 mass % to about 60 mass %, about 50 mass % to about 65 mass %, about 50 mass % to about 70 mass %, about 50 mass % to about 75 mass %, about 50 mass % to about 80 mass %, about 50 mass % to about 85 mass %, about 50 mass % to about 90 mass %, about 55 mass % to about 95 mass %, about 60 mass % to about 95 mass %, about 65 mass % to about 95 mass %, about 70 mass % to about 95 mass %, about 75 mass % to about 95 mass %, about 80 mass % to about 95 mass %, or about 85 mass % to about 95 mass % relative to the combined weight of the vitamin C and the coating formulation. In still further aspects, the choline may be present in an amount of about 50 mass %, 55 mass %, 60 mass %, 65 mass %, 70 mass %, 75 mass %, 80 mass %, 85 mass %, 90 mass %, or about 95 mass % relative to the combined mass of the vitamin C and the coating formulation. In some exemplary embodiments, the choline may be present in an amount from about 75 mass % and about 85 mass % relative to the combined mass of the choline and the coating formulation.

### C. Dosage Form

Further described herein are multi-ingredient dosage forms that include a coating formulation of the present disclosure and at least one active ingredient. The coating formulation of the present disclosure is used to coat the active ingredients described in Section IB above; however, the coating formulation preferably does not coat the entire dosage form. The multi-ingredient dosage form includes additional ingredients, including vitamins, minerals, nutrients, supplements, drugs, excipients, fillers, binders, lubricants, plasticizers, etc.

In some embodiments, the multi-ingredient dosage form may be a tablet, capsule, gummy, beverage, bar, suspension, granulation, powder, lozenge, troche, granule, pill, gel cap, or other dosage form known in the art. In examples wherein the dosage form is a tablet, the coating formulation may resist fracturing when the ingredients are compressed in the tablet.

The multi-ingredient dosage form may be modified to have a desired release profile. In some embodiments, the dosage form may be immediate release, controlled release, extended release, modified release, or sustained release.

The multi-ingredient dosage form includes a coating formulation of the present disclosure (see section IA of the detailed description herein). The coating formulation includes shellac. The coating formulation coats at least one active ingredient in the multi-ingredient dosage form (see section IB of the detailed description herein).

The at least one active ingredient may include vitamin C. The vitamin C may include ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, silver ascorbate, or any other forms of vitamin C known in the art or combinations thereof. In some examples, the vitamin C is ascorbic acid.

In some embodiments, the vitamin C may be present in an amount from about 50 mass % to about 95 mass % relative to the combined mass of the vitamin C and the coating formulation. In some aspects, the vitamin C may be present in an amount from about 50 mass % to about 55 mass %, about 55 mass % to about 60 mass %, about 60 mass % to about 65 mass %, about 65 mass % to about 70 mass %, about 70 mass % to about 75 mass %, about 75 mass % to about 80 mass %, about 80 mass % to about 85 mass %, about 85 mass % to about 90 mass %, or about 90 mass % to about 95 mass % relative to the combined mass of the vitamin C and the coating formulation. In some additional aspects, the vitamin C may be present in an amount from about 50 mass % to about 60 mass %, about 50 mass % to about 65 mass %, about 50 mass % to about 70 mass %, about 50 mass % to about 75 mass %, about 50 mass % to about 80 mass %, about 50 mass % to about 85 mass %, about 50 mass % to about 90 mass %, about 55 mass % to about 95 mass %, about 60 mass % to about 95 mass %, about 65 mass % to about 95 mass %, about 70 mass % to about 95 mass %, about 75 mass % to about 95 mass %, about 80 mass % to about 95 mass %, or about 85 mass % to about 95 mass % relative to the combined weight of the vitamin C and the coating formulation. In still further aspects, the vitamin C may be present in an amount of about 50 mass %, 55 mass %, 60 mass %, 65 mass %, 70 mass %, 75 mass %, 80 mass %, 85 mass %, 90 mass %, or about 95 mass % relative to the combined mass of the vitamin C and the coating formulation. In some exemplary embodiments, the vitamin C may be present in an amount from about 70 mass % to about 80 mass % relative to the combined mass of the vitamin C and the coating formulation.

In some embodiments, the vitamin C may be present in the multi-ingredient dosage form in an amount from about 1 mg to about 2000 mg. In some aspects, the vitamin C may be present in the multi-ingredient dosage form in an amount from about 1 mg to about 250 mg, about 250 mg to about 500 mg, about 500 mg to about 750 mg, about 750 mg to about 1000 mg, about 1000 mg to about 1250 mg, about 1250 mg to about 1500 mg, about 1500 mg to about 1750 mg, about 1750 mg to about 2000 mg. In some additional aspects, the vitamin C may be present in the multi-ingredient dosage form in an amount from about 1 mg to about 500 mg, about 1 mg to about 750 mg, about 1 mg to about 1000 mg, about 1 mg to about 1250 mg, about 1 mg to about 1500 mg, about 1 mg to about 1750 mg, about 250 mg to about 2000 mg, about 500 mg to about 2000 mg, about 750 mg to about 2000 mg, about 1000 mg to about 2000 mg, about 1250 mg to about 2000 mg, about 1500 mg to about 2000 mg, about 250 mg to about 1750 mg, about 500 mg to about 1500 mg, or about 750 mg to about 1250 mg. In still further aspects, the vitamin C may be present in the multi-ingredient dosage form in an amount of about 1 mg, 10 mg, 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or about 2000 mg.

The at least one active ingredient may include choline. The choline may include choline chloride, choline bitartrate, phosphatidyl choline, choline dihydrogen citrate, alpha glycerylphosphorylcholine, cytidine diphosphate-choline, choline folate, an ester of choline with DHA, esters of choline with 3-methyl-2-hexenoic acid (MHA), esters of choline with medium chain triglycerides, other esters of choline with fatty acids, other salts of choline, or any other forms of choline known in the art or combinations thereof. In some exemplary embodiments, the choline is choline bitartrate.

The choline may be present in an amount from about 50 mass % to about 95 mass % relative to the combined weight of the coating formulation and the choline. In some aspects, the choline may be present in an amount from about 50 mass % to about 55 mass %, about 55 mass % to about 60 mass %, about 60 mass % to about 65 mass %, about 65 mass % to about 70 mass %, about 70 mass % to about 75 mass %, about 75 mass % to about 80 mass %, about 80 mass % to about 85 mass %, about 85 mass % to about 90 mass %, or about 90 mass % to about 95 mass % relative to the combined mass of the coating formulation and the choline. In some additional aspects, the choline may be present in an amount from about 50 mass % to about 60 mass %, about 50 mass % to about 65 mass %, about 50 mass % to about 70 mass %, about 50 mass % to about 75 mass %, about 50 mass % to about 80 mass %, about 50 mass % to about 85 mass %, about 50 mass % to about 90 mass %, about 55 mass % to about 95 mass %, about 60 mass % to about 95 mass %, about 65 mass % to about 95 mass %, about 70 mass % to about 95 mass %, about 75 mass % to about 95 mass %, about 80 mass % to about 95 mass %, or about 85 mass % to about 95 mass % relative to the combined weight of the vitamin C and the coating formulation. In still further aspects, the choline may be present in an amount of about 50 mass %, 55 mass %, 60 mass %, 65 mass %, 70 mass %, 75 mass %, 80 mass %, 85 mass %, 90 mass %, or about 95 mass % relative to the combined mass of the vitamin C and the coating formulation. In some exemplary embodiments, the choline may be present in an amount between about 75 mass % and about 85 mass % relative to the combined mass of the choline and the coating formulation.

The choline may be present in the multi-ingredient dosage form in an amount of between about 1 mg to about 2000 mg. In some aspects, the choline may be present in the multi-ingredient dosage form in an amount from about 1 mg to about 250 mg, about 250 mg to about 500 mg, about 500 mg to about 750 mg, about 750 mg to about 1000 mg, about 1000 mg to about 1250 mg, about 1250 mg to about 1500 mg, about 1500 mg to about 1750 mg, about 1750 mg to about 2000 mg. In some additional aspects, the choline may be present in the multi-ingredient dosage form in an amount from about 1 mg to about 500 mg, about 1 mg to about 750 mg, about 1 mg to about 1000 mg, about 1 mg to about 1250 mg, about 1 mg to about 1500 mg, about 1 mg to about 1750 mg, about 250 mg to about 2000 mg, about 500 mg to about 2000 mg, about 750 mg to about 2000 mg, about 1000 mg to about 2000 mg, about 1250 mg to about 2000 mg, about 1500 mg to about 2000 mg, about 250 mg to about 1750 mg, about 500 mg to about 1500 mg, or about 750 mg to about 1250 mg. In still further aspects, the choline may be present in the multi-ingredient dosage form in an amount of about 1 mg, 10 mg, 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or about 2000 mg.

The multi-ingredient dosage form may additionally include vitamins other than vitamin C. In some embodiments, the vitamins may include one or more of vitamin A (i.e., retinoids or carotene), vitamin D (i.e., calciferol), vitamin E (i.e., alpha-tocopherol), vitamin K (i.e., phylloquinone or menadione), vitamin B1 (i.e., thiamin), vitamin B2 (i.e., riboflavin), vitamin B3 (i.e., niacin), vitamin B5 (i.e., panteothenic acid), vitamin B6 (i.e., pyridoxine), vitamin B7 (i.e., biotin), vitamin B12 (i.e., cyanocobalamin), vitamin B9 (i.e., folate or folic acid), or other vitamins known by those having ordinary skill in the art.

The multi-ingredient dosage form may additionally include minerals. In some embodiments, the minerals may include calcium, chloride, chromium, copper, fluoride, iodine, iron, magnesium, manganese, molybdenum, phosphorus, potassium, selenium, sodium, sulfur, zinc, or other minerals known by those having ordinary skill in the art or pharmaceutically acceptable salts thereof.

The multi-ingredient dosage form may additionally include supplements. In some embodiments, the supplements may include 5-hydroxytryptophan, antioxidants, amino acids, activated charcoal, ashwagandha, astaxanthin, astragalus, berberine, bromelain, chlorella, chlorophyll, chondroitin, coconut oil, cod liver oil, colloidal silver, conjugated linoleic acid, creatine, damiana, echinacea, emu oil, evening primrose oil, fenugreek, fiber, fish oil, gamma-aminobutyric acid, garlic, ginger, ginkgo, glucomannan, glucosamine, glutathione, gotu kola, hyaluronic acid, inositol, krill oil, licorice, lutein, maca, melatonin, moringa, omega fatty acids, prebiotics, probiotics, quercetin, rhodiola, saw palmetto, serrapeptase, taurine, turmeric, valerian, whey protein, xylitol, or other supplements known to those having ordinary skill in the art.

The multi-ingredient dosage form may additionally include excipients. The excipients may include binders, plasticizers, fillers, bulking agents, disintegrating agents, glidants, lubricants, diluents, flavoring agents, odorizing agents, flavor-masking agents, odor-masking agents, anti-caking agents, preservatives, humectants, wetting agents, and other excipients known to those having skill in the art.

The multi-ingredient dosage form may include one or more additional active ingredients that are not coated by the coating formulation described herein.

The dosage forms of the present disclosure may show little or no spotting after about 18 days of storage at a temperature of 25° C. and a relative humidity of 20%. Methods for measuring spotting are described in Section II below. For example, the dosage forms may show little or no spotting after about 18 days, 30 days, 60 days, 90 days, 120 days, 180 days, 250 days, or 300 days of storage at a temperature of 25° C. and a relative humidity of 20%. The amount of spotting may be determined by colorimeter measurements as described herein.

The dosage forms of the present disclosure may show little or no spotting after about 18 days of storage at a temperature of 45° C. and a relative humidity of 20%. For example, the dosage forms may show little or no spotting after about 18 days, 30 days, 60 days, 90 days, 120 days, 180 days, 250 days, or 300 days of storage at a temperature of 45° C. and a relative humidity of 20%. The amount of spotting may be determined by colorimeter measurements as described herein.

The dosage forms of the present disclosure may show little or no spotting after about 18 days of storage at a temperature of 25° C. and a relative humidity of 30%. For example, the dosage forms may show little or no spotting after about 18 days, 30 days, 60 days, 90 days, 120 days, 180 days, 250 days, or 300 days of storage at a temperature of 25° C. and a relative humidity of 30%. The amount of spotting may be determined by colorimeter measurements as described herein.

The dosage forms of the present disclosure may show little or no spotting after about 18 days of storage at a temperature of 45° C. and a relative humidity of 30%. For example, the dosage forms may show little or no spotting after about 18 days, 30 days, 60 days, 90 days, 120 days, 180 days, 250 days, or 300 days of storage at a temperature of 45° C. and a relative humidity of 30%. The amount of spotting may be determined by colorimeter measurements as described herein.

The multi-ingredient dosage form may be suitable for administration to a subject. The subject may a human, such as an adult or a child.

### II. Methods

### A. Methods of Preventing Spotting in a Dosage Form

Described herein is a method for preventing spotting in a dosage form containing vitamin C and/or choline. The method includes providing at least one active ingredient that includes at least one of vitamin C and choline, coating the at least one active ingredient in an coating formulation comprising shellac, and including the at least one active ingredient coated in the coating formulation in a dosage form. The coating formulation may be any coating formulation of the present disclosure described in Section I.

The step of coating the at least one active ingredient in a coating formulation comprising shellac may be accomplished through pan coating, fluid bed coating, spray blending, or other coating methods known in the art.

The amount of spotting in a dosage form may be determined by a visual determination or by colorimetric measurements. A colorimeter may be used to measure the amount of spotting in a dosage form by comparing it to a reference dosage form of the same composition. The color change may be measured by determining the total color difference (ΔE) of the dosage form compared to the reference dosage form, or by other methods known in the art, wherein a ΔE of 0 indicates no color change. In some aspects, the average ΔE in a dosage form may be about 2.5 or less, or more preferably about 1.0 or less. For example, the ΔE may be about 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or less than 0.1. A ΔE of less than 1.0 is generally considered a visually imperceptible color change. As used herein a dosage form with “little spotting” refers to a dosage form with a ΔE of 2.5 or less as compared to a reference dosage form; a dosage form with “no spotting” refers to a dosage form with a ΔE of 1.0 or less as compared to a reference dosage form.

The dosage forms of the present disclosure may show little or no spotting after about 18 days of storage at a temperature of 25° C. and a relative humidity of 20%. For example, the dosage forms may show little or no spotting after about 18 days, 30 days, 60 days, 90 days, 120 days, 180 days, 250 days, or 300 days of storage at a temperature of 25° C. and a relative humidity of 20%. The amount of spotting may be determined by colorimeter measurements as described herein.

The dosage forms of the present disclosure may show little or no spotting after about 18 days of storage at a temperature of 45° C. and a relative humidity of 20%. For example, the dosage forms may show little or no spotting after about 18 days, 30 days, 60 days, 90 days, 120 days, 180 days, 250 days, or 300 days of storage at a temperature of 45° C. and a relative humidity of 20%. The amount of spotting may be determined by colorimeter measurements as described herein.

The dosage forms of the present disclosure may show little or no spotting after about 18 days of storage at a temperature of 25° C. and a relative humidity of 30%. For example, the dosage forms may show little or no spotting after about 18 days, 30 days, 60 days, 90 days, 120 days, 180 days, 250 days, or 300 days of storage at a temperature of 25° C. and a relative humidity of 30%. The amount of spotting may be determined by colorimeter measurements as described herein.

The dosage forms of the present disclosure may show little or no spotting after about 18 days of storage at a temperature of 45° C. and a relative humidity of 30%. For example, the dosage forms may show little or no spotting after about 18 days, 30 days, 60 days, 90 days, 120 days, 180 days, 250 days, or 300 days of storage at a temperature of 45° C. and a relative humidity of 30%. The amount of spotting may be determined by colorimeter measurements as described herein.

### B. Methods of Making a Coating Formulation and Dosage Forms

The coating formulations of the present disclosure may be manufactured by methods known in the art. The coating formulation may be made by dissolving the shellac and other coating formulation ingredients described in Section I above in a suitable solvent, such as an alcohol solvent. In an embodiment, the solvent may be ethanol.

Next, the active ingredient may be provided in a particulate form (e.g., a granule). The active ingredient may include choline or vitamin C, or any other active ingredient provided in Section I.

The coating formulation may then be applied to the active ingredient using coating methods known in the art, such as pan coating, spray coating, etc. When the solvent dries, the coating formulation is applied to the active ingredient, thus forming a coated composition. The coated composition may be sieved to adjust the average particle size of the coated composition.

The dosage forms may be manufactured by various methods known to those having skill in the art. The dosage form may be tablet, capsule, gummy, beverage, bar, suspension, granulation, powder, lozenge, troche, granule, pill, gel cap or other solid dosage forms known to those having skill in the art. In an exemplary embodiment, the dosage form is a tablet. The coated compositions may be incorporated into the dosage forms using methods known to those having ordinary skill in the art. For example, the coated composition may be combined with other ingredients and compressed to form a tablet. It will be appreciated that some unit operations may be modified or excluded to prevent damage to the coating formulation. For example, milling the dosage form may be excluded to prevent damaging the coating formulation.

### C. Methods of Administration

Further provided herein are methods of administering the dosage forms described in Section I. The methods may include orally, sublingually, or buccally administering a dosage form of the present disclosure to a subject. The subject may a human, such as an adult or a child. In some aspects, the subject may have a nutritive deficiency or insufficiency, such as a vitamin C deficiency, a choline deficiency, or both. Thus, in some aspects, the methods may be suitable for supplementing nutrition to a subject.

### EXEMPLARY EMBODIMENTS

Embodiment 1: A coating formulation comprising shellac, wherein the coating formulation coats at least one active ingredient in a multi-ingredient dosage form.

Embodiment 2: The coating formulation of embodiment 1, wherein the at least one active ingredient has a concentration of between about 50.0 mass % to about 95.0 mass % relative to the total mass of the at least one active ingredient and the coating formulation.

Embodiment 3: The coating formulation of embodiment 1 or embodiment 2, wherein the at least one active ingredient comprises vitamin C.

Embodiment 4: The coating formulation of embodiment 3, wherein the vitamin C is selected from the group consisting of ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, silver ascorbate, and any combination thereof.

Embodiment 5: The coating formulation of embodiment 3 or embodiment 4, wherein the vitamin C is present in an amount from about 50 mass % to about 95 mass % relative to the combined mass of the vitamin C and the coating formulation.

Embodiment 6: The coating formulation of any one of embodiments 1-5, wherein the at least one active ingredient comprises choline.

Embodiment 7: The coating formulation of claim 6, wherein the choline is selected from the group consisting of choline chloride, choline bitartrate, phosphatidyl choline, choline dihydrogen citrate, alpha glycerylphosphorylcholine, cytidine diphosphate-choline, choline folate, an ester of choline with DHA, esters of choline with 3-methyl-2-hexenoic acid (MHA), esters of choline with medium chain triglycerides, other esters of choline with fatty acids, other salts of choline, or combinations thereof.

Embodiment 8: The coating formulation of embodiment 6 or embodiment 7, wherein the choline is present in an amount from about 50 mass % to about 95 mass % relative to the combined mass of the choline and the coating formulation.

Embodiment 9: The coating formulation of any one of embodiments 1-8, wherein the shellac is present in an amount from about 1 mass % to about 50 mass % relative to the combined mass of the at least one active ingredient and the coating formulation.

Embodiment 10: The coating formulation of any one of embodiments 1-9, wherein the coating formulation further comprises a silicate.

Embodiment 11: The coating formulation of embodiment 10, wherein the silicate includes silicon dioxide, bentonite, magnesium aluminum silicate, magnesium silicate, magnesium trisilicate, silicified microcrystalline cellulose, or combinations thereof.

Embodiment 12: The coating formulation of embodiment 10 or embodiment 11, wherein the silicate is present in the coating formulation in an amount from about 0 mass % to about 10 mass % relative to the combined mass of the coating formulation and the at least one active ingredient.

Embodiment 13: The coating formulation of embodiment 11, wherein the silicon dioxide includes colloidal silicon dioxide or fumed silica.

Embodiment 14: The coating formulation of any one of embodiments 1-13, wherein the coating formulation further comprises cellulose or a derivative thereof.

Embodiment 15: The coating formulation of embodiment 14, wherein the cellulose or a derivative thereof includes microcrystalline cellulose, powdered cellulose, hydroxypropylcellulose, hydroxypropylmethylcellulose, hydroxypropylcellulose, methylcellulose, ethylcellulose, hydroxyethylcellulose, hydroxyethylmethylcellulose, hypromellose, carboxymethylcellulose, or combinations thereof.

Embodiment 16: The coating formulation of embodiment 14 or embodiment 15, wherein the cellulose or a derivative thereof comprises hydroxypropylcellulose.

Embodiment 17: The coating formulation of embodiment 16, wherein the hydroxypropylcellulose is present in the coating formulation in an amount from about 0 mass % to about 10 mass % relative to the combined mass of the coating formulation and the at least one active ingredient.

Embodiment 18: The coating formulation of embodiment 14 or embodiment 15, wherein the cellulose or a derivative thereof comprises microcrystalline cellulose.

Embodiment 19: The coating formulation of embodiment 18, wherein the microcrystalline cellulose is present in the coating formulation in an amount from about 0 mass % to about 25 mass % relative to the combined mass of the coating formulation and the first active ingredient and second active ingredient.

Embodiment 20: The coating formulation of embodiment 14 or embodiment 15, wherein the cellulose or a derivative thereof comprises ethylcellulose.

Embodiment 21: The coating formulation of embodiment 20, wherein the ethylcellulose is present in the coating formulation in an amount from about 0 mass % to about 10 mass % relative to the combined mass of the coating formulation and the first active ingredient or the second active ingredient.

Embodiment 22: The coating formulation of any one of embodiments 1-21, wherein the coating formulation further comprises starch.

Embodiment 23: The coating formulation of embodiment 22, wherein the starch includes maize starch, potato starch, rice starch, wheat starch, tapioca starch, or combinations thereof.

Embodiment 24: The coating formulation of embodiment 22 or embodiment 23, wherein the starch is present in the coating formulation in an amount from about 0 mass % to about 25 mass % relative to the combined mass of the coating formulation and the first active ingredient or the second active ingredient.

Embodiment 25: A multi-ingredient dosage form, the dosage form comprising: at least one active ingredient; and a coating formulation comprising shellac, wherein the coating formulation coats the at least one active ingredient.

Embodiment 26: The dosage form of embodiment 25, wherein the at least one active ingredient comprises vitamin C.

Embodiment 27: The dosage form of embodiment 26, wherein the vitamin C is present in the dosage form in an amount of about 1 mg to about 2000 mg.

Embodiment 28: The dosage form of embodiment 26 or embodiment 27, wherein the vitamin C is selected from the group consisting of ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, silver ascorbate, and any combination thereof.

Embodiment 29: The dosage form of any one of embodiments 25-28, wherein the at least one active ingredient comprises choline.

Embodiment 30: The dosage form of embodiment 29, wherein the choline is present in the dosage form in an amount from about 1 mg to about 2000 mg.

Embodiment 31: The dosage form of embodiment 29 or embodiment 30, wherein the choline is selected from the group consisting of choline chloride, choline bitartrate, phosphatidyl choline, choline dihydrogen citrate, alpha glycerylphosphorylcholine, cytidine diphosphate-choline, choline folate, an ester of choline with DHA, esters of choline with 3-methyl-2-hexenoic acid (MHA), esters of choline with medium chain triglycerides, other esters of choline with fatty acids, other salts of choline, or combinations thereof.

Embodiment 32: The dosage form of any one of embodiments 25-31, wherein the shellac is present in an amount from about 1 mass % to about 50 mass % relative to the total mass of the at least one active ingredient and the coating formulation.

Embodiment 33: The dosage form of any one of embodiments 25-32, wherein the at least one active ingredient is present in an amount from about 50.0 mass % to about 95.0 mass % relative to the combined mass of the at least one active ingredient and the coating formulation.

Embodiment 34: The dosage form of any one of embodiments 25-33, wherein the at least one active ingredient comprises vitamin C and choline, and wherein the choline is not coated by the coating formulation.

Embodiment 35: The dosage form of any one of embodiments 25-33, wherein the at least one active ingredient comprises choline and the dosage form further comprises vitamin C, wherein the vitamin C is not coated by the coating formulation.

Embodiment 36: The dosage form of any one of embodiments 25-33, wherein the at least one active ingredient comprises vitamin C and the dosage form further comprises choline, wherein the choline is not coated by the coating formulation.

Embodiment 37: The dosage form of any one of embodiments 25-36, wherein the dosage form is a tablet, capsule, gummy, beverage, bar, suspension, granulation, powder, lozenge, troche, granule, pill, gel cap.

Embodiment 38: The dosage form of any one of embodiments 25-37, wherein the dosage form further comprises at least one of vitamins, minerals, supplements, and excipients.

Embodiment 39: The dosage form of any one of embodiments 25-38, wherein the dosage form further comprises an additional active ingredient that is that is not coated by the coating formulation.

Embodiment 40: The dosage form of any one of embodiments 25-39, wherein the coating formulation further comprises a silicate.

Embodiment 41: The dosage form of embodiment 40, wherein the silicate includes silicon dioxide, bentonite, magnesium aluminum silicate, magnesium silicate, magnesium trisilicate, silicified microcrystalline cellulose, or combinations thereof.

Embodiment 42: The dosage form of embodiment 40 or embodiment 41, wherein the silicate is present in the coating formulation in an amount from about 0 mass % to about 10 mass % relative to the combined mass of the coating formulation and the at least one active ingredient.

Embodiment 43: The dosage form of embodiment 41, wherein the silicon dioxide includes colloidal silicon dioxide or fumed silica.

Embodiment 44: The dosage form of any one of embodiments 25-43, wherein the coating formulation further comprises cellulose or a derivative thereof.

Embodiment 45: The dosage form of embodiment 44, wherein the cellulose or a derivative thereof includes microcrystalline cellulose, powdered cellulose, hydroxypropylcellulose, hydroxypropylmethylcellulose, hydroxypropylcellulose, methylcellulose, ethylcellulose, hydroxyethylcellulose, hydroxyethylmethylcellulose, hypromellose, carboxymethylcellulose, or combinations thereof.

Embodiment 46: The dosage form of embodiment 44 or embodiment 45, wherein the cellulose or a derivative thereof comprises hydroxypropylcellulose.

Embodiment 47: The dosage form of embodiment 46, wherein the hydroxypropylcellulose is present in the coating formulation in an amount from about 0 mass % to about 10 mass % relative to the combined mass of the coating formulation and the at least one active ingredient.

Embodiment 48: The dosage form of embodiment 44 or embodiment 45, wherein the cellulose or a derivative thereof comprises microcrystalline cellulose.

Embodiment 49: The dosage form of embodiment 48, wherein the microcrystalline cellulose is present in the coating formulation in an amount from about 0 mass % to about 25 mass % relative to the combined mass of the coating formulation and the first active ingredient and second active ingredient.

Embodiment 50: The dosage form of embodiment 44 or embodiment 45, wherein the cellulose or a derivative thereof comprises ethylcellulose.

Embodiment 51: The dosage form of embodiment 50, wherein the ethylcellulose is present in the coating formulation in an amount from about 0 mass % to about 10 mass % relative to the combined mass of the coating formulation and the first active ingredient or the second active ingredient.

Embodiment 52: The dosage form of any one of embodiments 25-51, wherein the coating formulation further comprises starch.

Embodiment 53: The dosage form of embodiment 52, wherein the starch includes maize starch, potato starch, rice starch, wheat starch, tapioca starch, or combinations thereof.

Embodiment 54: The dosage form of embodiment 52 or embodiment 53, wherein the starch is present in the coating formulation in an amount from about 0 mass % to about 25 mass % relative to the combined mass of the coating formulation and the first active ingredient or the second active ingredient.

Embodiment 55: The dosage form of any one of embodiments 25-54, wherein the multi-ingredient dosage form shows little spotting after storage for about 18 days at 25° C. and 20% relative humidity.

Embodiment 56: The dosage form of any one of embodiments 25-55, wherein the multi-ingredient dosage form shows little spotting after storage for about 60 days at 25° C. and 20% relative humidity.

Embodiment 57: The dosage form of any one of embodiments 25-56, wherein the multi-ingredient dosage form shows little spotting after storage for about 120 days at 25° C. and 20% relative humidity.

Embodiment 58: The dosage form of any one of embodiments 25-57, wherein the multi-ingredient dosage form shows little spotting after storage for about 18 days at 45° C. and 20% relative humidity.

Embodiment 59: The dosage form of any one of embodiments 25-58, wherein the multi-ingredient dosage form shows little spotting after storage for about 60 days at 45° C. and 20% relative humidity.

Embodiment 60: The dosage form of any one of embodiments 25-59, wherein the multi-ingredient dosage form shows little spotting after storage for about 120 days at 45° C. and 20% relative humidity.

Embodiment 61: The dosage form of any one of embodiments 25-60, wherein the multi-ingredient dosage form shows little spotting after storage for about 18 days at 25° C. and 30% relative humidity.

Embodiment 62: The dosage form of any one of embodiments 25-61, wherein the multi-ingredient dosage form shows little spotting after storage for about 60 days at 25° C. and 30% relative humidity.

Embodiment 63: The dosage form of any one of embodiments 25-62, wherein the multi-ingredient dosage form shows little spotting after storage for about 120 days at 25° C. and 30% relative humidity.

Embodiment 64: The dosage form of any one of embodiments 25-63, wherein the multi-ingredient dosage form shows little spotting after storage for about 18 days at 45° C. and 30% relative humidity.

Embodiment 65: The dosage form of any one of embodiments 25-64, wherein the multi-ingredient dosage form shows little spotting after storage for about 60 days at 45° C. and 30% relative humidity.

Embodiment 66: The dosage form of any one of embodiments 25-65, wherein the multi-ingredient dosage form shows little spotting after storage for about 120 days at 45° C. and 30% relative humidity.

Embodiment 67: A multi-ingredient dosage form, the dosage form comprising: choline; vitamin C; and a coating formulation comprising shellac, wherein the coating formulation coats the choline.

Embodiment 68: A multi-ingredient dosage form, the dosage form comprising: choline; vitamin C; and a coating formulation comprising shellac, wherein the coating formulation coats the vitamin C.

Embodiment 69: A multi-ingredient dosage form, the dosage form comprising: choline, wherein the choline is coated by a first coating formulation comprising shellac; and vitamin C, wherein the vitamin C is coated by a second coating formulation comprising shellac.

Embodiment 70: A method of preventing spotting in a dosage form, the method comprising: providing an active ingredient comprising at least one of vitamin C and choline; coating the active ingredient in a coating formulation comprising shellac; and including the active ingredient coated in the coating formulation in a dosage form.

Embodiment 71: The method of embodiment 70, wherein the active ingredient is vitamin C.

Embodiment 72: The method of embodiment 70 or embodiment 71, wherein the active ingredient is choline.

Embodiment 73: The method of any one of embodiments 70-72, wherein the active ingredient is choline and vitamin C.

Embodiment 74: The method of any one of embodiments 70-73, wherein the coating formulation further comprises a silicate.

Embodiment 75: The method of embodiment 74, wherein the silicate includes silicon dioxide, bentonite, magnesium aluminum silicate, magnesium silicate, magnesium trisilicate, silicified microcrystalline cellulose, or combinations thereof.

Embodiment 76: The method of embodiment 74 or embodiment 75, wherein the silicate is present in the coating formulation in an amount from about 0 mass % to about 10 mass % relative to the combined mass of the coating formulation and the at least one active ingredient.

Embodiment 77: The method of embodiment 75, wherein the silicon dioxide includes colloidal silicon dioxide or fumed silica.

Embodiment 78: The method of any one of embodiments 70-77, wherein the coating formulation further comprises cellulose or a derivative thereof.

Embodiment 79: The method of embodiment 78, wherein the cellulose or a derivative thereof includes microcrystalline cellulose, powdered cellulose, hydroxypropylcellulose, hydroxypropylmethylcellulose, hydroxypropylcellulose, methylcellulose, ethylcellulose, hydroxyethylcellulose, hydroxyethylmethylcellulose, hypromellose, carboxymethylcellulose, or combinations thereof.

Embodiment 80: The method of embodiment 78 or embodiment 79, wherein the cellulose or a derivative thereof comprises hydroxypropylcellulose.

Embodiment 81: The method of embodiment 80, wherein the hydroxypropylcellulose is present in the coating formulation in an amount from about 0 mass % to about 10 mass % relative to the combined mass of the coating formulation and the at least one active ingredient.

Embodiment 82: The method of embodiment 78 or embodiment 79, wherein the cellulose or a derivative thereof comprises microcrystalline cellulose.

Embodiment 83: The method of embodiment 82, wherein the microcrystalline cellulose is present in the coating formulation in an amount from about 0 mass % to about 25 mass % relative to the combined mass of the coating formulation and the first active ingredient and second active ingredient.

Embodiment 84: The method of embodiment 78 or embodiment 79, wherein the cellulose or a derivative thereof comprises ethylcellulose.

Embodiment 85: The method of embodiment 84, wherein the ethylcellulose is present in the coating formulation in an amount from about 0 mass % to about 10 mass % relative to the combined mass of the coating formulation and the first active ingredient or the second active ingredient.

Embodiment 86: The method of any one of embodiments 70-85, wherein the coating formulation further comprises starch.

Embodiment 87: The method of embodiment 86, wherein the starch includes maize starch, potato starch, rice starch, wheat starch, tapioca starch, or combinations thereof.

Embodiment 88: The method of embodiment 86 or embodiment 87, wherein the starch is present in the coating formulation in an amount from about 0 mass % to about 25 mass % relative to the combined mass of the coating formulation and the first active ingredient or the second active ingredient.

Embodiment 89: The method of any one of embodiments 70-88, wherein the multi-ingredient dosage form shows little spotting after storage for about 18 days at 25° C. and 20% relative humidity.

Embodiment 90: The method of any one of embodiments 70-89, wherein the multi-ingredient dosage form shows little spotting after storage for about 60 days at 25° C. and 20% relative humidity.

Embodiment 91: The method of any one of embodiments 70-90, wherein the multi-ingredient dosage form shows little spotting after storage for about 120 days at 25° C. and 20% relative humidity.

Embodiment 92: The method of any one of embodiments 70-91, wherein the multi-ingredient dosage form shows little spotting after storage for about 18 days at 45° C. and 20% relative humidity.

Embodiment 93: The method of any one of embodiments 70-92, wherein the multi-ingredient dosage form shows little spotting after storage for about 60 days at 45° C. and 20% relative humidity.

Embodiment 94: The method of any one of embodiments 70-93, wherein the multi-ingredient dosage form shows little spotting after storage for about 120 days at 45° C. and 20% relative humidity.

Embodiment 95: The method of any one of embodiments 70-94, wherein the multi-ingredient dosage form shows little spotting after storage for about 18 days at 25° C. and 30% relative humidity.

Embodiment 96: The method of any one of embodiments 70-95, wherein the multi-ingredient dosage form shows little spotting after storage for about 60 days at 25° C. and 30% relative humidity.

Embodiment 97: The method of any one of embodiments 70-96, wherein the multi-ingredient dosage form shows little spotting after storage for about 120 days at 25° C. and 30% relative humidity.

Embodiment 98: The method of any one of embodiments 70-97, wherein the multi-ingredient dosage form shows little spotting after storage for about 18 days at 45° C. and 30% relative humidity.

Embodiment 99: The method of any one of embodiments 70-98, wherein the multi-ingredient dosage form shows little spotting after storage for about 60 days at 45° C. and 30% relative humidity.

Embodiment 100: The method of any one of embodiments 70-99, wherein the multi-ingredient dosage form shows little spotting after storage for about 120 days at 45° C. and 30% relative humidity.

### EXAMPLES

### Example 1: Exemplary Dosage Form

A dosage form of the present disclosure was made that included the following ingredients: vitamin A, vitamin D, biotin, magnesium stearate, vitamin K1, thiamine, riboflavin, niacin, vitamin B6, folic acid, vitamin B12, pantothenic acid, calcium, magnesium oxide, potassium chloride, iodine, phosphorous, ferrous fumarate, zinc oxide, copper oxide, manganese sulfate, molybdenum, chromium, picolinate, choline bitartrate, and vitamin C. This formulation was used to test different coating formulations of the present disclosure. The formulation was modified in some cases to include choline. When the formulation did not include choline, hydroxypropyl cellulose was included in the formulation in the same amount, i.e., 280 mg. The amounts of each component in the formulation are shown in Table 1.

TABLE 1

| Ingredients | Amount | unit |
|---|---|---|
| Vitamin A as Betacarotene, Palmitate | 2500 | IU |
| Vitamin D (Cholecalcepherol) | 400 | IU |
| Biotin (Vit H) | 75 | mcg |
| Magnesium Stearate | 12 | mg |
| Vitamin K1 (HPC) | 20 | mcg |
| Thiamine B1 | 0.75 | mg |
| Riboflavin B2 | 0.85 | mg |
| Niacin B3 (Niacinamide) | 10 | mg |
| Vitamin B6 | 1 | mg |
| Folic Acid | 0.2 | mg |
| Vitamin B12 | 3 | mcg |
| Pantothenic Acid as calcium (vit B5) | 5 | mg |
| Calcium (Dicalcium Phosphate) | 100 | mg |
| Magnesium Oxide | 40 | mg |
| Potassium chloride | 70 | mg |
| Potassium iodide | 75 | mcg |
| Phosphorus (Dicalcium Phosphate) | 100 | mg |
| Ferrous Fumarate | 82 | mg |
| Zinc Oxide | 3.75 | mg |
| Copper Oxide | 0.4 | mg |
| Manganese Sulfate | 0.5 | mg |
| Molybdenum (as sodium) | 18.75 | mcg |
| Chromium Picolinate | 12 | mcg |
| Choline Bitartrate (optional) | 280 | mg |
| Vitamin C (Ascorbic Acid) (optional) | 120 | mg |

FIG. shows a tablet that contained vitamin C but no choline under different storage conditions after 18 days. The tablet composition is shown in Table 2. The storage conditions ranged from 10% relative humidity (RH) to 30% RH, and 25° C. to 45° C. Spotting occurred under each storage condition to some degree, with the most noticeable spotting occurring at high temperature and humidity. One tablet stored at 40° C. and 75% RH exhibited severe spotting after only 24 hours.

TABLE 2

| Ingredients | Amount | unit |
|---|---|---|
| Vitamin A as Betacarotene, Palmitate | 2500 | IU |
| Vitamin D (Cholecalcepherol) | 400 | IU |
| Biotin (Vit H) | 75 | mcg |
| Magnesium Stearate | 12 | mg |
| Vitamin K1 (HPC) | 20 | mcg |
| Thiamine B1 | 0.75 | mg |
| Riboflavin B2 | 0.85 | mg |
| Niacin B3 (Niacinamide) | 10 | mg |
| Vitamin B6 | 1 | mg |
| Folic Acid | 0.2 | mg |
| Vitamin B12 | 3 | mcg |
| Pantothenic Acid as calcium (vit B5) | 5 | mg |
| Calcium (Dicalcium Phosphate) | 100 | mg |
| Magnesium Oxide | 40 | mg |
| Potassium chloride | 70 | mg |
| Potassium iodide | 75 | mcg |
| Phosphorus (Dicalcium Phosphate) | 100 | mg |
| Ferrous Fumarate | 82 | mg |
| Zinc Oxide | 3.75 | mg |
| Copper Oxide | 0.4 | mg |
| Manganese Sulfate | 0.5 | mg |
| Molybdenum (as sodium) | 18.75 | mcg |
| Chromium Picolinate | 12 | mcg |
| Hydroxypropyl Cellulose | 280 | mg |
| Vitamin C (Ascorbic Acid) | 120 | mg |

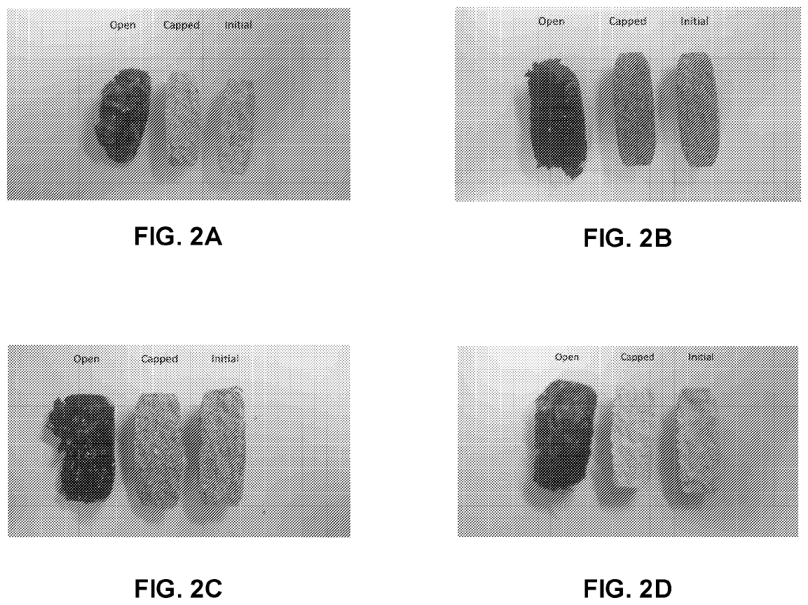

FIGS. 2A-2D shows commercially available tablets that were stored in different conditions for 18 days. The tablets in FIGS. 2A, 2B, and 2D did not contain any choline, whereas the tablet in FIG. 2C contained 30 mg of choline. All tablets contained vitamin C. The tablets were cut in half for the experiment. The tablet halves labelled “open” and “initial” were two halves of the same tablet, and the tablet labelled “capped” was a half of a separate tablet. The tablets labelled “open” were stored in at 40° C. and 75% RH for 24 hours. The tablets labelled “capped” were also stored at 40° C. and 75% RH for 24 hours but were covered with a cap inside the humidity chamber. The tablets labelled “initial” were stored at room temperature and 0% RH. Each tablet showed severe spotting in the “open” conditions.

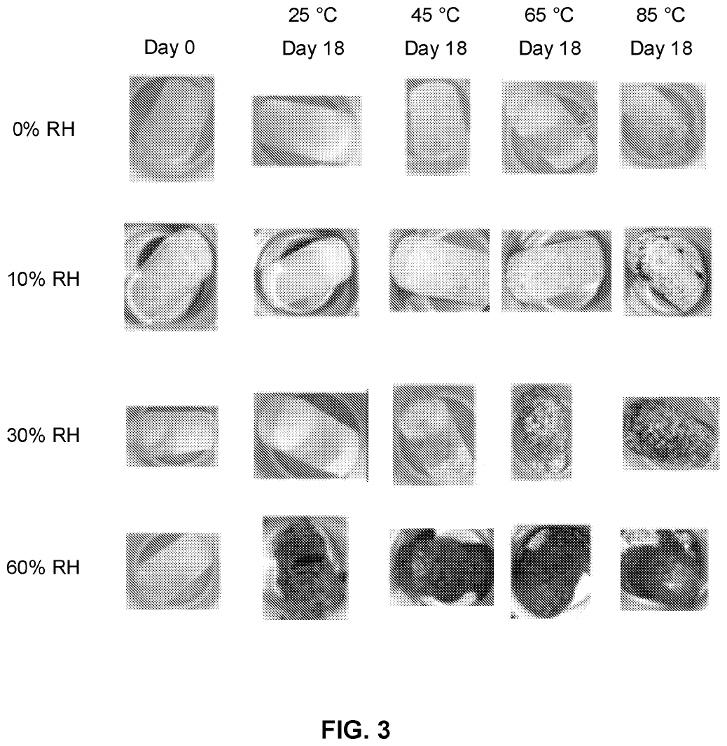

FIG. 3 shows the spotting for a tablet containing choline and vitamin C stored under different temperature and humidity conditions for 18 days. The tablet composition is shown in Table 3. The temperature conditions tested were 25° C., 45° C., 65° C., and 85° C., and the relative humidity conditions tested were 0%, 10%, 30%, and 60%. As can be seen from the figure, spotting accelerated as temperature and humidity increased. At 60% relative humidity, the tablet was almost entirely black after 18 days, regardless of the temperature.

TABLE 3

| Ingredients | Amount | unit |
|---|---|---|
| Vitamin A as Betacarotene, Palmitate | 2500 | IU |
| Vitamin D (Cholecalcepherol) | 400 | IU |
| Biotin (Vit H) | 75 | mcg |
| Magnesium Stearate | 12 | mg |
| Vitamin K1 (HPC) | 20 | mcg |
| Thiamine B1 | 0.75 | mg |
| Riboflavin B2 | 0.85 | mg |
| Niacin B3 (Niacinamide) | 10 | mg |
| Vitamin B6 | 1 | mg |
| Folic Acid | 0.2 | mg |
| Vitamin B12 | 3 | mcg |
| Pantothenic Acid as calcium (vit B5) | 5 | mg |
| Calcium (Dicalcium Phosphate) | 100 | mg |
| Magnesium Oxide | 40 | mg |
| Potassium chloride | 70 | mg |
| Potassium iodide | 75 | mcg |
| Phosphorus (Dicalcium Phosphate) | 100 | mg |
| Ferrous Fumarate | 82 | mg |
| Zinc Oxide | 3.75 | mg |
| Copper Oxide | 0.4 | mg |
| Manganese Sulfate | 0.5 | mg |
| Molybdenum (as sodium) | 18.75 | mcg |
| Chromium Picolinate | 12 | mcg |
| Choline Bitartrate | 280 | mg |
| Vitamin C (Ascorbic Acid) | 120 | mg |

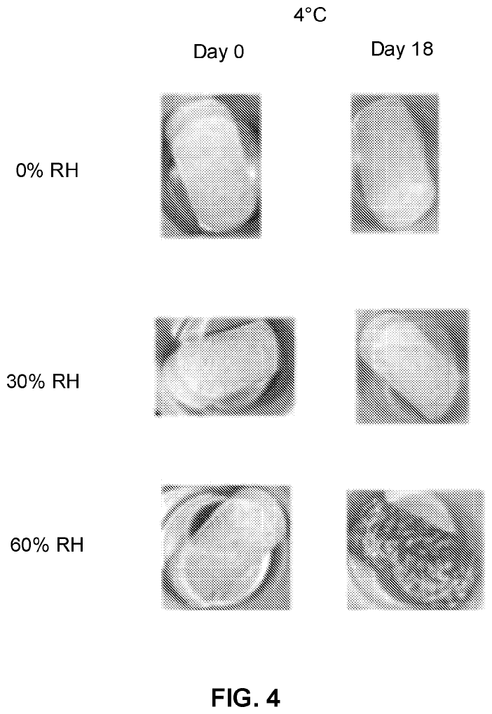

FIG. 4 shows spotting for a tablet containing choline and vitamin C under refrigerated conditions. The tablet had the composition of the tablet described in Table 3. The tablets were stored at 4° C. for 18 days at 0% RH, 30% RH, and 60% RH. After 18 days, even in refrigerated conditions, the tablet stored at 60% RH still exhibited severe spotting.

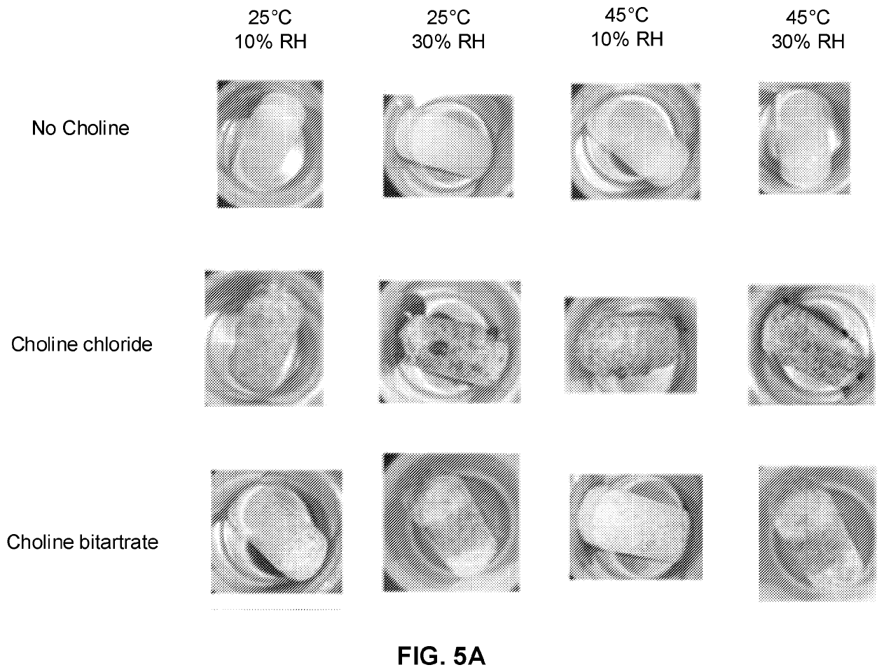

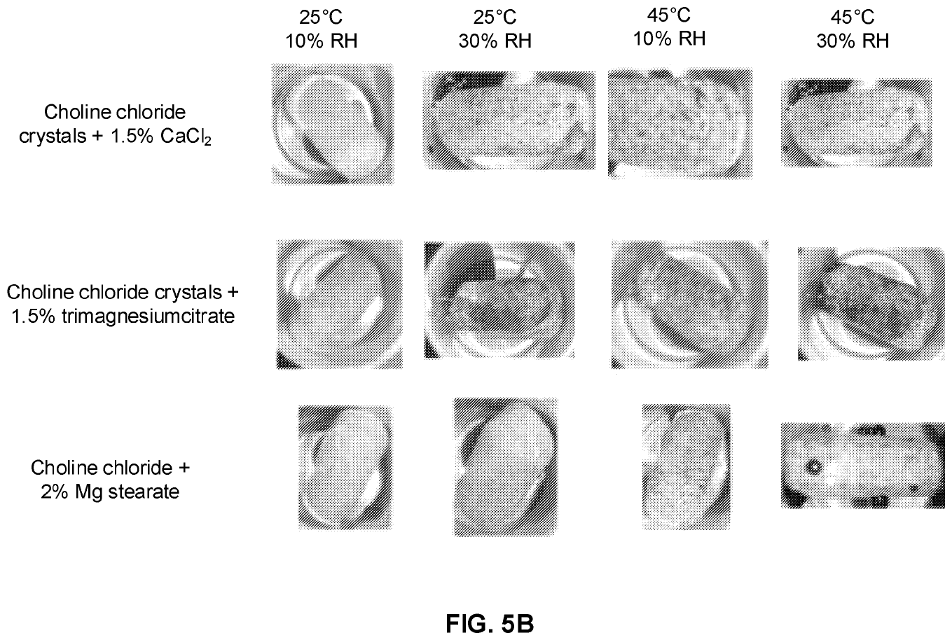

FIGS. 5A and 5B show spotting for a tablet containing vitamin C and various forms of choline as compared to a tablet containing no choline. The general tablet composition is described in Table 4. The tablets were stored for 18 days at temperatures of either 25° C. or 45° C. and a relative humidity of either 10% or 30%. One tablet containing no choline was used as a reference. FIG. 5A shows the reference tablet and tablets containing choline chloride and choline bitartrate. FIG. 5B shows tablets containing choline chloride crystals blended with 1.5% calcium chloride, choline chloride crystals blended with 1.5% trimagnesiumcitrate, and choline chloride blended with 2% magnesium stearate. As used herein and through the remainder of Example 1, “choline chloride crystals blended with 1.5% calcium chloride” means that the choline chloride crystals are blended with 1.5% w/w of calcium chloride in relation to the combined mass of the choline chloride and the coating. Stated another way, every 98.5 mg of choline chloride in the tablet is blended with 1.5 mg of calcium chloride. As can clearly be seen from FIGS. 5A-5B, spotting occurred under nearly all storage conditions with the addition of the various forms of choline. Additionally, the figures suggest that generally the relative humidity has a large effect on the degree of spotting.

TABLE 4

| Ingredients | Amount | unit |
|---|---|---|
| Vitamin A as Betacarotene, Palmitate | 2500 | IU |
| Vitamin D (Cholecalcepherol) | 400 | IU |
| Biotin (Vit H) | 75 | mcg |
| Magnesium Stearate | 12 | mg |
| Vitamin K1 (HPC) | 20 | mcg |
| Thiamine B1 | 0.75 | mg |
| Riboflavin B2 | 0.85 | mg |
| Niacin B3 (Niacinamide) | 10 | mg |
| Vitamin B6 | 1 | mg |
| Folic Acid | 0.2 | mg |
| Vitamin B12 | 3 | mcg |
| Pantothenic Acid as calcium (vit B5) | 5 | mg |
| Calcium (Dicalcium Phosphate) | 100 | mg |
| Magnesium Oxide | 40 | mg |
| Potassium chloride | 70 | mg |
| Potassium iodide | 75 | mcg |
| Phosphorus (Dicalcium Phosphate) | 100 | mg |
| Ferrous Fumarate | 82 | mg |
| Zinc Oxide | 3.75 | mg |
| Copper Oxide | 0.4 | mg |
| Manganese Sulfate | 0.5 | mg |
| Molybdenum (as sodium) | 18.75 | mcg |
| Chromium Picolinate | 12 | mcg |
| Choline | 280 | mg |
| Vitamin C (Ascorbic Acid) | 120 | mg |

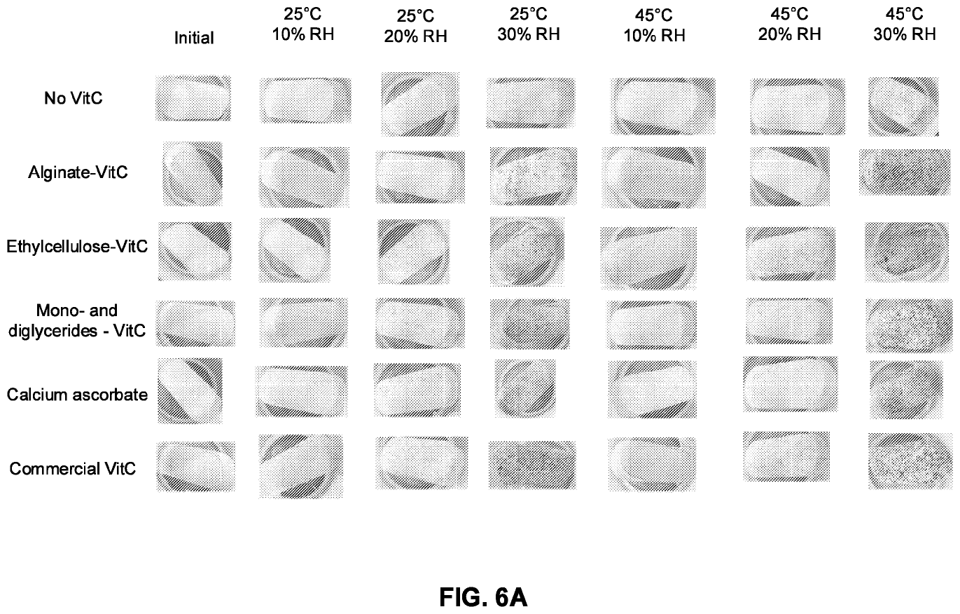

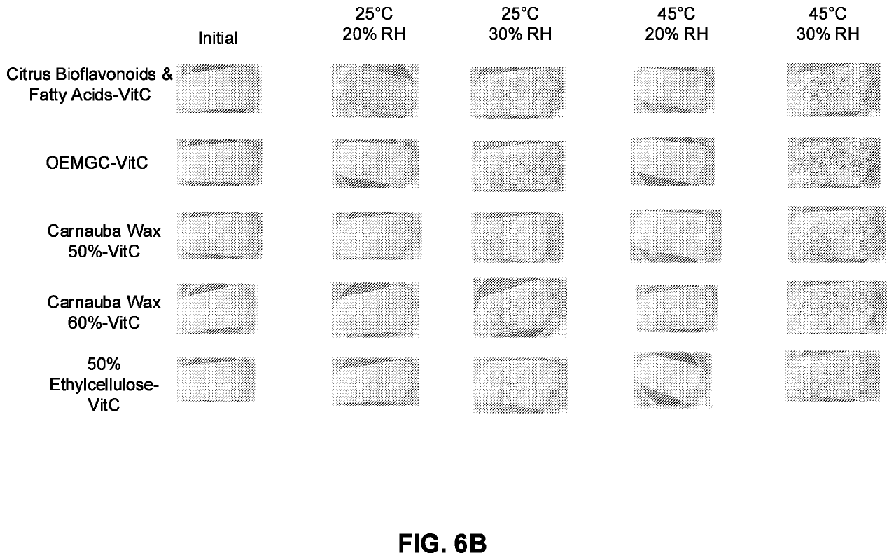

FIG. 6A shows spotting for a tablet including various forms of vitamin C (VitC) and choline in the form of choline bitartrate. The general composition of the tablets is provided in Table 5. A tablet containing no vitamin C is used as a reference. The tablets were stored for 18 days at temperatures of 25° C. or 45° C. and at a relative humidity of 10%, 20%, or 30%. The tablets tested in order from top to bottom of FIG. 6A included vitamin C in a matrix of alginate beads, vitamin C in a matrix of ethylcellulose, vitamin C in a matrix of vegetable mono- and diglycerides (60% vitamin C), vitamin C in the form of calcium ascorbate, and a commercial vitamin C formulation containing ascorbic acid, soybean oil, and silicon dioxide. As expected from the previous trials, increased temperature and relative humidity increased the degree of spotting for all of the various vitamin C forms. Some degree of spotting occurred at all temperature and humidity conditions.

TABLE 5

| Ingredients | Amount | unit |
|---|---|---|
| Vitamin A as Betacarotene, Palmitate | 2500 | IU |
| Vitamin D (Cholecalcepherol) | 400 | IU |
| Biotin (Vit H) | 75 | mcg |
| Magnesium Stearate | 12 | mg |
| Vitamin K1 (HPC) | 20 | mcg |
| Thiamine B1 | 0.75 | mg |
| Riboflavin B2 | 0.85 | mg |
| Niacin B3 (Niacinamide) | 10 | mg |
| Vitamin B6 | 1 | mg |
| Folic Acid | 0.2 | mg |
| Vitamin B12 | 3 | mcg |
| Pantothenic Acid as calcium (vit B5) | 5 | mg |
| Calcium (Dicalcium Phosphate) | 100 | mg |
| Magnesium Oxide | 40 | mg |
| Potassium chloride | 70 | mg |
| Potassium iodide | 75 | mcg |
| Phosphorus (Dicalcium Phosphate) | 100 | mg |
| Ferrous Fumarate | 82 | mg |
| Zinc Oxide | 3.75 | mg |
| Copper Oxide | 0.4 | mg |
| Manganese Sulfate | 0.5 | mg |
| Molybdenum (as sodium) | 18.75 | mcg |
| Chromium Picolinate | 12 | mcg |
| Choline Bitartrate | 280 | mg |
| Vitamin C | 120 | mg |

FIG. 6B shows spotting for a tablet containing vitamin C and choline and various coating formulations. The tablet compositions are described in Table 3. The tablets were stored for 18 days at temperatures of 25° C. or 45° C. and at a relative humidity of 20% or 30%. The tablets tested in order from top to bottom of FIG. 6B included vitamin C blended with 3.8% citrus bioflavonoids and 3.3% fatty acids, vitamin C coated in a mixture of 0.13% oleic acid, 1.12% ethyl cellulose, 0.02% medium-chain triglycerides, 0.03% glycerin, and 1% calcium stearate (OEMGC), vitamin C blended with 50% carnauba wax (CB), vitamin C blended with 60% carnauba wax (CB), and vitamin C blended with ethylcellulose. Although the spotting was greatly reduced in comparison to previous trials, the tablets still exhibited some degree of spotting.

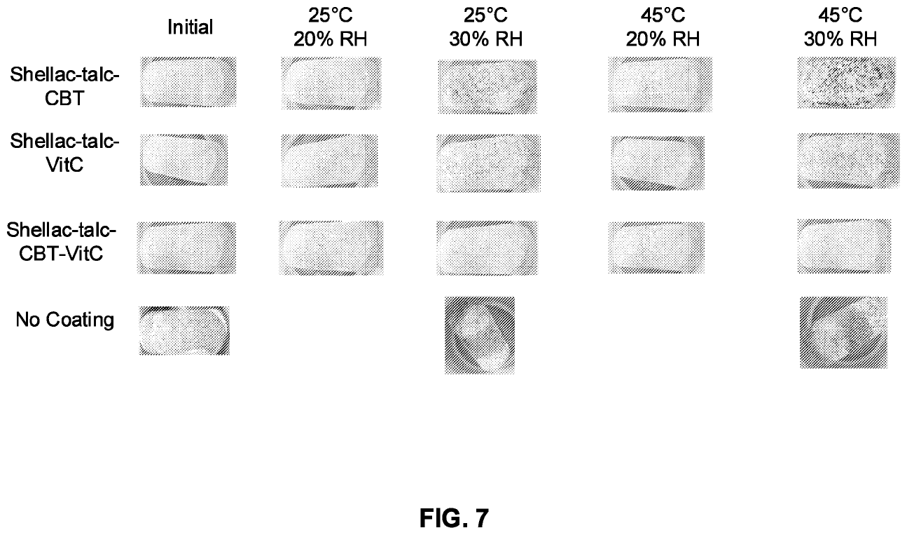

FIG. 7 shows spotting for a tablet containing both vitamin C and choline and various coating formulations. The tablet compositions are described in Table 3. The tablets were stored for 18 days at temperatures of 25° C. or 45° C. and at a relative humidity of 20% or 30%. The tablets tested in order from top to bottom of FIG. 7 included a tablet containing choline bitartrate coated with a mixture of shellac and talc, a tablet containing vitamin C coated with a mixture of shellac and talc, a tablet containing vitamin C and choline bitartrate both coated in a mixture of shellac and talc, and a tablet containing choline bitartrate and vitamin C without a coating. It is clear from FIG. 7 that there was a reduction in spotting when the vitamin C alone was coated with shellac compared to the tablet containing choline bitartrate coated in shellac. However, the tablet wherein both vitamin C and choline bitartrate were coated with shellac showed little to no spotting.

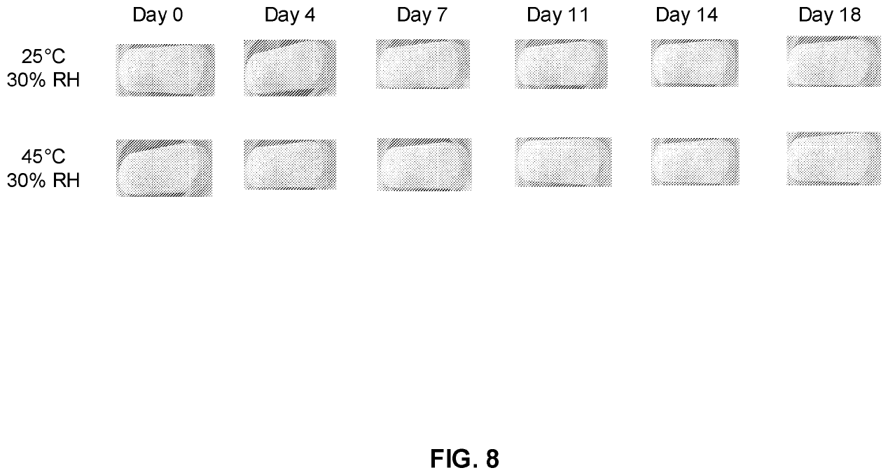

FIG. 8 shows the progression of spotting for a tablet containing both vitamin C and choline and coating formulations that included a mixture of shellac and talc. The tablet compositions are described in Table 3. The tablets were stored for 18 days at temperatures of 25° C. or 45° C. and at a relative humidity of 30%. No spotting was observed for any of the tablets. However, due to regulation, talc cannot be used for certain pharmaceutical applications. Thus, more coating formulations were developed that did not contain talc to be used for pharmaceutical applications.

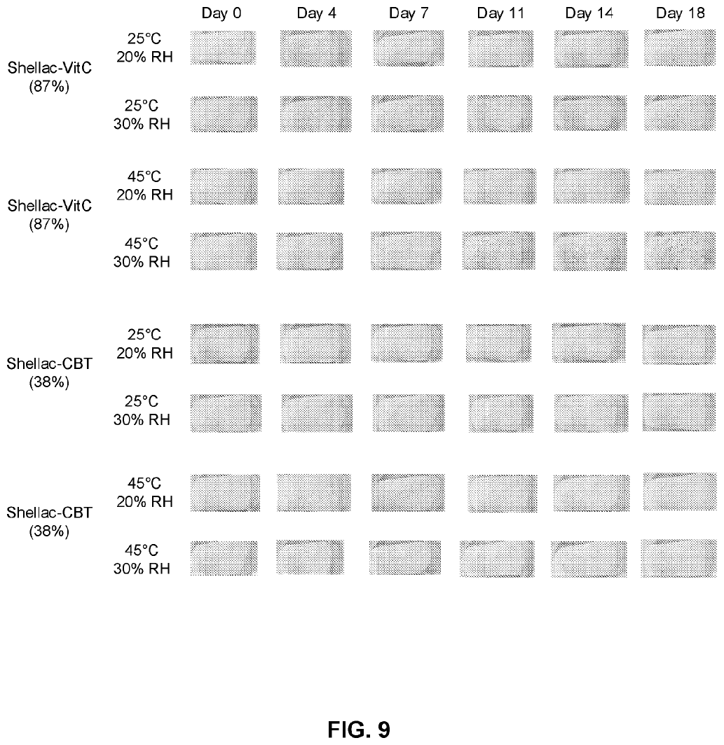

FIG. 9 shows the progression of spotting for a tablet containing shellac coatings on vitamin C or on choline bitartrate. The tablet compositions are described in Table 3. The percentages refer to the weight percent of the active ingredient relative to the combined weight of the active ingredient and the coating; i.e., the tablet labeled “Shellac-VitC (87%)” means that the tablet contained 87% vitamin C by weight and 13% coating by weight. The shellac coating did not contain any other additives such as talc; i.e., the coating contained only shellac. The tablets were stored for 18 days at temperatures of 25° C. or 45° C. and at a relative humidity of 20% or 30%. The images show the tablets on days 0, 4, 7, 11, 14, and 18. The tablets containing 87% vitamin C coated in shellac showed spotting at only high temperature and humidity. However, the remaining tablets showed little to no spotting.

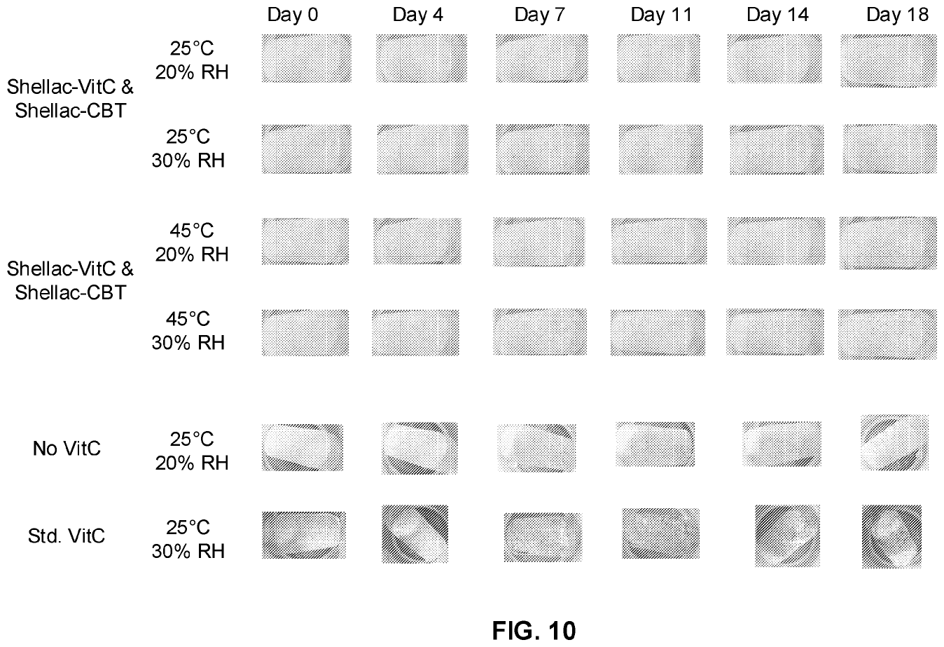

FIG. 10 shows the progression of spotting for a tablet containing shellac coatings on vitamin C or on choline bitartrate. The tablet compositions are described in Table 3. The tablets were stored for 18 days at temperatures of 25° C. or 45° C. and at a relative humidity of 20% or 30%. The images show the tablets on days 0, 4, 7, 11, 14, and 18. Tablets that included no vitamin C and uncoated ascorbic acid were included for reference. The tablets that included shellac-coated vitamin C and choline bitartrate showed little to no spotting.

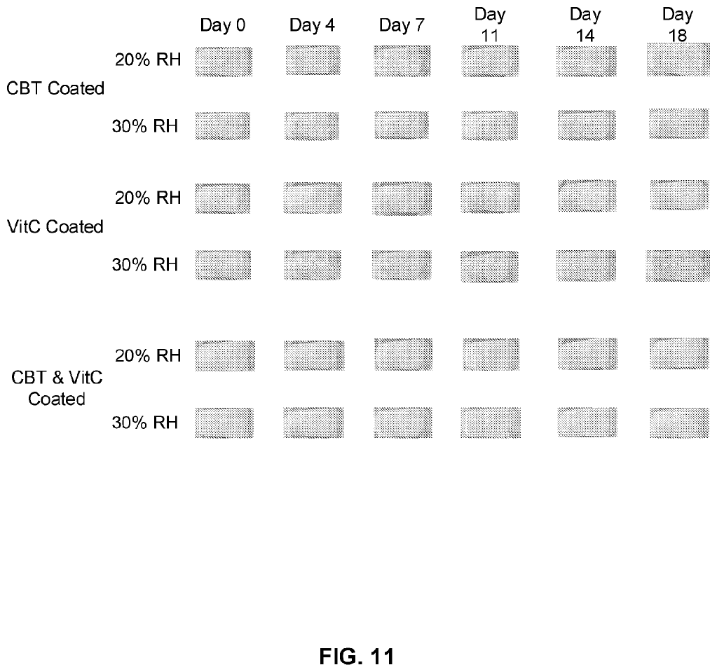

FIG. 11 shows the progression of spotting for a tablet containing shellac coatings on vitamin C or on choline bitartrate, or on both. The tablet compositions are described in Table 3. The tablets were stored for 18 days at a temperature of 45° C. and at a relative humidity of 20% or 30%. The images show the tablets on days 0, 4, 7, 11, 14, and 18. It can clearly be seen that tablets that contained choline bitartrate and vitamin C coated in shellac showed no spotting. Moreover, tablets that contained either choline bitartrate coated in shellac or vitamin C coated in shellac still exhibited little or no spotting.

Coating formulations were made to coat choline bitartrate and ascorbic acid (vitamin C) within a dosage form. Table 6 shows different formulations that were made.

TABLE 6

| | | | | | |
|---|---|---|---|---|---|
| | Coating 1 | Coating 2 | Coating 3 | Coating 4 | Coating 5 |
| Ingredient | (mass %) | (mass %) | (mass %) | (mass %) | (mass %) |

Active Ingredient

| | | | | | |
|---|---|---|---|---|---|
| Choline | 76.15 | 79.84 | 79.86 | N/A | N/A |
| Bitartrate | | | | | |
| Vitamin C | N/A | N/A | N/A | 70.04 | 70.84 |
| Shellac | 5.77 | 8.06 | 7.10 | 17.35 | 17.71 |

Coating Ingredients

| | | | | | |
|---|---|---|---|---|---|
| Colloidal | 0.92 | 2.0 | 1.53 | 1.51 | 1.37 |
| Silicon | | | | | |
| Dioxide | | | | | |
| Maize Starch | 15.33 | 10.10 | N/A | 11.10 | N/A |
| Micro- | N/A | N/A | 10.81 | N/A | 10.08 |
| crystalline | | | | | |
| Cellulose | | | | | |
| Hydroxy- | 1.15 | N/A | 0.42 | N/A | N/A |
| propyl- | | | | | |
| cellulose | | | | | |
| Ethyl- | 0.78 | N/A | 0.28 | N/A | N/A |
| cellulose | | | | | |

### Example 2: Colorimeter Measurements

A HunterLab UltraScan VIS colorimeter was used to perform colorimetric analysis on the tablets. One or more of each tablet tested was reserved to be used as the initial colorimeter measurement. The reserved tablet(s) was stored in the dark in a desiccator to prevent spotting.

Tablets were ground to a powder using a mortar and pestle. The powder was then transferred into a 100 mL volumetric amber flask. 80-90 mL of distilled water was added to the flask. The flask was capped and the contents were shaken several times. Then, the flask was filled to the 100 mL mark with distilled water and shaken again.

Next, 25 mL of the liquid from the flask was transferred to a 50 mL centrifuge tube. The tube was spun at 3200 rpm for 10 minutes. The liquid was decanted and poured into a clean colorimeter cuvette. The cuvette was placed into the colorimeter and the color spectrum of the liquid was measured. The reserve tablet was measured first and saved as a reference or origin. The total color difference (ΔE) was calculated. A ΔE of less than one is generally an imperceptible color change and is thus most desirable.

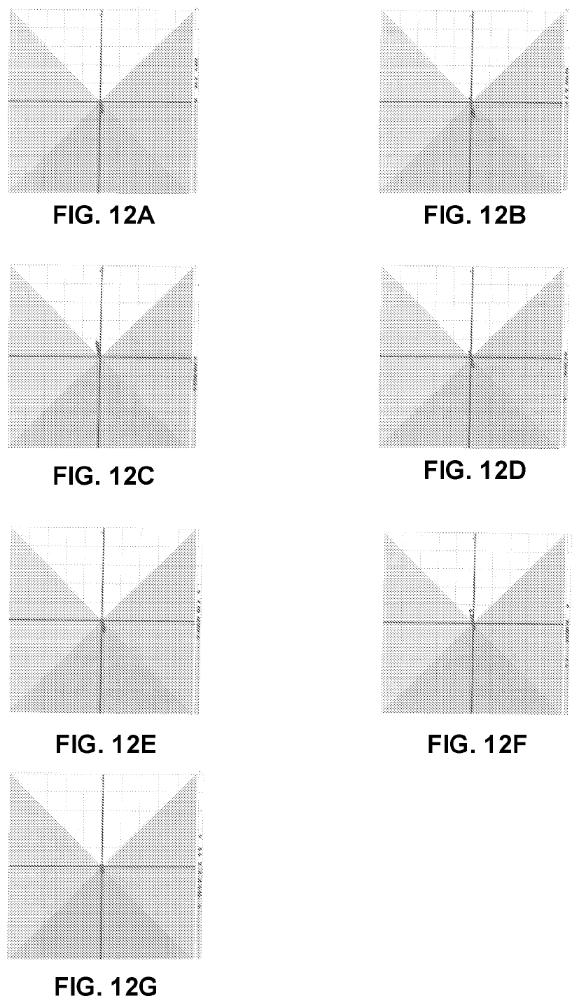

FIGS. 12A-12G show the colorimeter measurements for tablets containing a coating formulation of the present disclosure. All tablets were stored at 30% RH for 18 days. FIG. 12A shows the colorimeter measurements from a tablet containing coating formulations 1 and 4 described in Table 6. FIG. 12B shows the colorimeter measurements from a tablet containing coating formulations 1 and 5 described in Table 6. FIG. 12C shows the colorimeter measurements from a tablet containing coating formulations 2 and 4 described in Table 6. FIG. 12D shows the colorimeter measurements from a tablet containing coating formulations 2 and 5 described in Table 6. FIG. 12E shows the colorimeter measurements from a tablet containing coating formulations 3 and 4 described in Table 6. FIG. 12F shows the colorimeter measurements from a tablet containing coating formulations 3 and 5 described in Table 6. FIG. 12G shows the colorimeter measurements from a tablet containing coating formulations 1 and 5 described in Table 6, plus talc. Colorimeter measurements were conducted at various times over the course of 18 days.

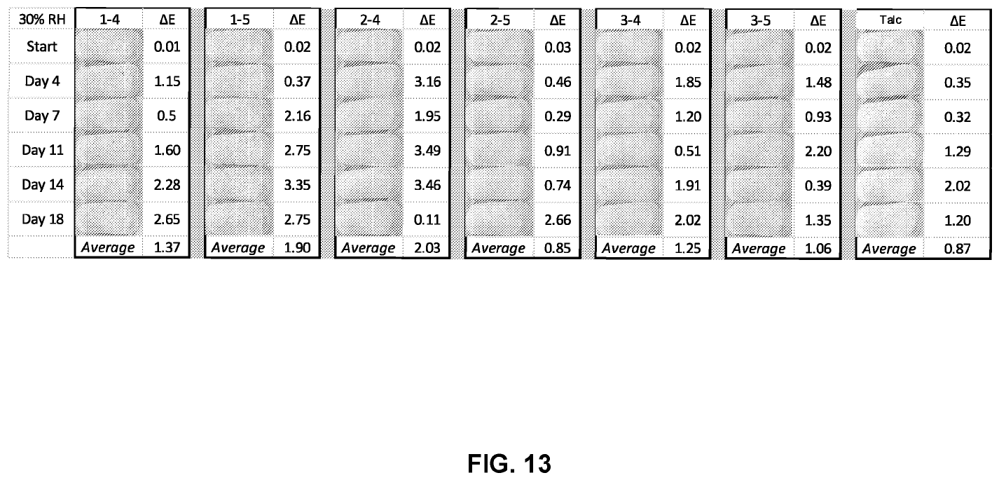

FIG. 13 shows the each of the tablets measured in FIGS. 12A-12G, the ΔE measured on days 0, 4, 7, 11, 14, and 18, as well as the average ΔE measured for each tablet. Overall, the average ΔE ranged from 0.85 to 2.03, indicating a low degree of color change among the tablets.

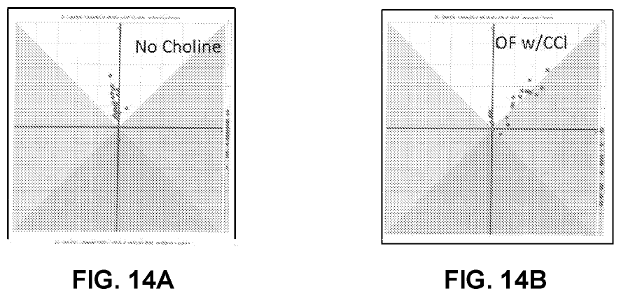

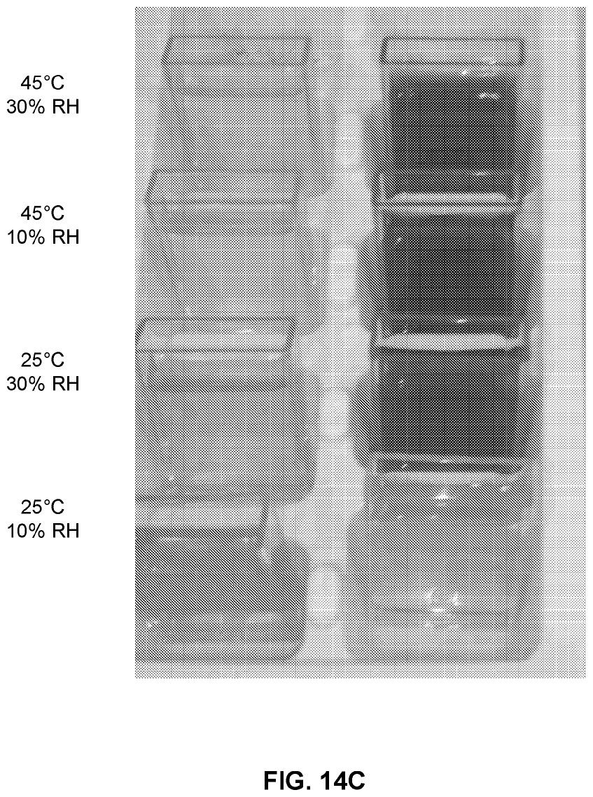

FIGS. 14A-14B show colorimeter measurements for tablets that did not contain coating formulations of the present disclosure. FIG. 14A shows the colorimeter measurements for a tablet that contained vitamin C and no choline. FIG. 14B shows the colorimeter measurements from a tablet containing vitamin C and choline chloride. It is clear that the tablets measured in FIGS. 14A-14B showed far greater color change compared to the tablets shown in FIGS. 12A-12G. Generally, tablets without a coating formulation of the present disclosure had an average ΔE of about 7-10. In one tablet that included choline chloride coated with calcium chloride, the ΔE on day 18 was 68.7, showing a large color change from the reference tablet.

FIG. 14C shows the solutions used for the colorimeter experiments of FIGS. 14A-14B after 18 days. The cuvettes on the left side are the samples for tablets that contained vitamin C and no choline. The cuvettes on the right side are the samples for tablets that contained vitamin C and choline chloride.

1. A coating formulation comprising shellac, wherein the coating formulation coats at least one active ingredient in a multi-ingredient dosage form.

2. The coating formulation of claim 1, wherein the at least one active ingredient has a concentration of between about 50.0 mass % to about 95.0 mass % relative to the total mass of the at least one active ingredient and the coating formulation.

3. The coating formulation of claim 1, wherein the at least one active ingredient comprises vitamin C.

4. The coating formulation of claim 3, wherein the vitamin C is ascorbic acid, ferrous ascorbate, magnesium ascorbate, sodium ascorbate, calcium ascorbate, zinc ascorbate, molybdenum ascorbate, chromium ascorbate, manganese ascorbate, lithium ascorbate, potassium ascorbate, nickel ascorbate, or silver ascorbate, or a combination thereof.

5. The coating formulation of claim 3, wherein the vitamin C is present in an amount from about 50 mass % to about 95 mass % relative to the combined mass of the vitamin C and the coating formulation.

6. The coating formulation of claim 1, wherein the at least one active ingredient comprises choline.

7. The coating formulation of claim 6, wherein the choline is choline chloride, choline bitartrate, phosphatidyl choline, choline dihydrogen citrate, alpha glycerylphosphorylcholine, cytidine diphosphatecholine, choline folate, an ester of choline with DHA, an ester of choline with 3-methyl-2-hexenoic acid (MHA), an ester of choline with medium chain triglycerides, or an ester of choline with a fatty acid, or a combination thereof.

8. The coating formulation of claim 6, wherein the choline is present in an amount from about 50 mass % to about 95 mass % relative to the combined mass of the choline and the coating formulation.

9. The coating formulation of claim 1, wherein the shellac is present in an amount from about 1 mass % to about 50 mass % relative to the combined mass of the at least one active ingredient and the coating formulation.

10. The coating formulation of claim 1, wherein the coating formulation further comprises a silicate, cellulose or a derivative thereof, starch.

11. The coating formulation of claim 10, wherein the silicate is silicon dioxide, bentonite, magnesium aluminum silicate, magnesium silicate, magnesium trisilicate, or silicified microcrystalline cellulose, or a combination thereof.

12. The coating formulation of claim 10, wherein the silicate is present in the coating formulation in an amount from about 0 mass % to about 10 mass % relative to the combined mass of the coating formulation and the at least one active ingredient.

13. The coating formulation of claim 11, wherein the silicon dioxide is colloidal silicon dioxide or fumed silica.

14. (canceled)

15. The coating formulation of claim 10, wherein the cellulose or a derivative thereof is microcrystalline cellulose, powdered cellulose, hydroxypropylcellulose, hydroxypropylmethylcellulose, hydroxypropylcellulose, methylcellulose, ethylcellulose, hydroxyethylcellulose, hydroxyethylmethylcellulose, hypromellose, or carboxymethylcellulose, or a combination thereof.

16. The coating formulation of claim 15, wherein the cellulose or a derivative thereof is hydroxypropylcellulose, microcrystalline cellulose, or ethylcellulose.

17. The coating formulation of claim 16, wherein (i) the hydroxypropylcellulose or ethylcellulose is present in the coating formulation in an amount from about 0 mass % to about 10 mass % relative to the combined mass of the coating formulation and the at least one active ingredient or (ii) the microcrystalline cellulose is present in the coating formulation in an amount from about 0 mass % to about 25 mass % relative to the combined mass of the coating formulation and the first active ingredient and second active ingredient.

18-22. (canceled)

23. The coating formulation of claim 10, wherein the starch is maize starch, potato starch, rice starch, wheat starch, or tapioca starch, or a combination thereof.

24. The coating formulation of claim 23, wherein the starch is present in the coating formulation in an amount from about 0 mass % to about 25 mass % relative to the combined mass of the coating formulation and the first active ingredient or the second active ingredient.

25. A multi-ingredient dosage form, the dosage form comprising:
at least one active ingredient; and
the coating formulation of claim 1,
wherein the coating formulation coats the at least one active ingredient.

26. (canceled)

27. The multi-ingredient dosage form of claim 25, wherein the vitamin C and/or choline is present in the dosage form in an amount of about 1 mg to about 2000 mg.

28-33. (canceled)

34. The multi-ingredient dosage form claim 25, wherein:
(i) the at least one active ingredient comprises vitamin C and choline, and wherein the choline is not coated by the coating formulation; or
(ii) the at least one active ingredient comprises choline and the dosage form further comprises vitamin C, wherein the vitamin C is not coated by the coating formulation; or
(iii) the at least one active ingredient comprises vitamin C and the dosage form further comprises choline, wherein the choline is not coated by the coating formulation.

35-54. (canceled)

55. The multi-ingredient dosage form of claim 25, wherein the multi-ingredient dosage form has little spotting after storage for about 18, about 60, or about 120 days at 25° C. and 20% relative humidity, 45° C. and 20% relative humidity, 25° C. and 30% relative humidity, or 45° C. and 30% relative humidity.

56-66. (canceled)

67. The multi-ingredient dosage form of claim 25, comprising:
choline;
vitamin C; and
a coating formulation comprising shellac,
wherein the coating formulation coats the choline or vitamin C.

68. (canceled)

69. The multi-ingredient dosage form of claim 25, comprising:
choline, wherein the choline is coated by a first coating formulation comprising shellac; and
vitamin C, wherein the vitamin C is coated by a second coating formulation comprising shellac.

70. A method of preventing black spotting on a dosage form, the method comprising:
providing an active ingredient comprising at least one of vitamin C and choline;
coating the active ingredient in a coating formulation comprising shellac; and
including the active ingredient coated in the coating formulation in a dosage form.

71-100. (canceled)
